(19) United States
(12) Patent Application Publication
(10) Pub. No.: US 2026/0231953 A1
(43) Pub. Date: Aug. 13, 2026

(54) Epichloe Endophyte

(71) Applicant: Grasslanz Technology Limited, Lincoln (NZ)

(72) Inventors: Alan Vincent Stewart, Christchurch (NZ); David Edward Hume, Palmerston North (NZ); Wade Jeffray Mace, Palmerston North (NZ); Alison Jean Popay, Hamilton (NZ)

(21) Appl. No.: 18/271,400

(22) PCT Filed: Jan. 19, 2021
(86) PCT No.: PCT/IB2021/052670

§ 371 Date: Jul. 7, 2023

(30) Foreign Application Priority Data
Jan. 19, 2021 (NZ) 771857

(51) Int. Cl. A01N 63/30; A01P 3/00; C12N 1/145; C12R 1/645

(52) U.S. Cl. CPC A01N 63/30; A01P 3/00; C12N 1/145; C12R 2001/645

(57) ABSTRACT

Epichloë endophytes that produce bioactive secondary metabolites in symbiotic association with a host plant, particularly a grass host plant. Also disclosed are methods of using these endophytes to confer pest resistance on host plants, and combinations comprising these endophytes and host plants or parts thereof, including seeds.

### RELATED APPLICATIONS

This application is a U.S. National Stage application under 35 U.S.C. § 371 from International Patent Application No. PCT/IB2021/052670 filed Mar. 31, 2021, which claims the benefit of priority from New Zealand Patent Application No. 771857 filed Jan. 19, 2021.

### TECHNICAL FIELD

The present invention generally relates to Epichloë endophytes that produce bioactive secondary metabolites in symbiotic association with host plants, particularly grass plants. The invention also generally relates to methods of using the endophytes to confer pest resistance on host plants, and to combinations comprising these endophytes and host plants or parts thereof, including seeds.

### REFERENCE TO SEQUENCE LISTING

The Sequence Listing submitted as an XML filed named “1064205US 2025 ST25.txt,” created on Aug. 5, 2025, and having a size of 8.93 kB is hereby incorporated by reference pursuant to 37 C.F.R. § 1.835 (a) (2).

### BACKGROUND OF THE INVENTION

Lolium perenne, commonly known as ryegrass, is a major forage grass in New Zealand.

Lolium perenne in New Zealand is found naturally infected with asexual wild-type symbiotic Epichloë fungal endophytes (e.g., Epichloë festucae var. lolii) (‘wild-type’ is also commonly referred to as ‘standard endophyte’ or ‘common toxic endophyte’). Epichloë festucae var. lolii mediates the production, in planta, of various types of secondary metabolite, typically alkaloidal compounds, a number of which confer beneficial properties to their ryegrass hosts. In particular, at least three different types of alkaloid are produced in planta by the wild-type endophyte (ergot alkaloids, pyrrolopyrazines, and indole diterpenes), which are known to improve resistance of the host plant to various insect pests.

Ergovaline is an ergot alkaloid compound produced in planta within ryegrass infected with wild-type strains of Epichloë festucae var. lolii as well as with certain commercial strains. Ergovaline has been shown to provide ryegrass with resistance to adult black beetle (Heteronychus arator), a major pasture pest in northern New Zealand (Ball et al., 1997a).

Epichloë endophytes that colonise ryegrass are also known to produce the alkaloid peramine in planta. Peramine has been shown to confer insect protection against the Argentine stem weevil (Listronotus bonariensis), a major pasture pest in New Zealand (Rowan et al., 1990).

Rare examples of Epichloë festucae var. lolii (recently classified also as Epichloë LpTG-3 (Lolium perenne Taxonomic Group 3) (Hettiarachchige et al., 2015)) in planta also produce epoxyjanthitrems. These indole diterpene compounds produced by the endophyte in planta in ryegrass are associated with improved resistance of the host plant to porina (Wiseana cervinata) (Finch et al., 2020).

Additionally, Epichloë endophytes produce lolitrem alkaloids, including lolitrem B in planta in ryegrass. Lolitrem B is known to reduce the larval growth of Argentine stem weevil (Dymock et al., 1989).

The potential benefits of different levels and/or different types of these various alkaloids for the agricultural production of forage grasses and subsequently, the production of livestock grazing on those forage grasses, were recognized in the 1980s and 1990s. This recognition led to the development and commercialization of a number of Epichloë endophyte/plant associations, symbioses in which the endophyte strains produced, in planta, varying types and amounts of alkaloids which confer various levels of biotic and/or abiotic resistance to the host plant.

Unfortunately, certain alkaloids produced in planta by Epichloë endophytes (both wild-type and commercial strains) in ryegrass in New Zealand can have detrimental effects on animals that consume the infected plants as feed.

Heat stress, for example, including the associated symptoms of reduced weight gain, higher body temperature and increased respiration rate, is caused by above average concentrations of ergovaline present in ryegrass grazed by livestock.

Another detrimental consequence of Epichloë endophyte presence within forage ryegrass is the neurological impairment “ryegrass staggers”, known to affect New Zealand livestock since the early 1900s. Ryegrass staggers are caused by consumption of the tremorgenic mycotoxin lolitrem B produced by the endophyte in planta (Gallagher et al., 1981; Gallagher et al., 1982).

Importantly, ryegrass grown in New Zealand that is not infected with a selected or wild-type Epichloë endophyte is susceptible to attack by a number of different insect pests, including Argentine stem weevil, which is a major pasture pest (Popay and Hume, 2011; Prestidge et al., 1982; Mortimer and di Menna, 1983).

Accordingly, there is a need in the art for strains of Epichloë endophyte that will form symbioses with ryegrass plant hosts and produce levels of certain alkaloids, in planta, that will confer on the host plant, advantages in terms of insect pest resistance as described above, while avoiding some of the disadvantages associated with the in planta production of alkaloids that have detrimental effects on livestock.

It is an object of the present invention to provide at least one Epichloë endophyte strain which when combined with a host plant confers the benefits of at least some level of pest protection and/or disease protection on the host plant without causing ryegrass staggers and/or that at least reduces the severity of any ryegrass staggers compared to other known Epichloë endophytes, and/or to at least provide the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

In a first aspect, the present invention relates to an isolated strain of Epichloë endophyte wherein the endophyte comprises an ans017 allele size of 306±0.8 base pairs (bp), an ans030 allele size of 308±0.8 bp, an ans033 allele size of 171±0.8 bp, an ans054 allele size of 297±0.8 bp, an egs002 allele size of 297±0.8 bp and an egs027 allele size of 344±0.8 bp.

In another aspect, the invention relates to an isolated strain of Epichloë endophyte that is AR128 (NRRL 68010) or AR166 (NRRL 68011) or a combination thereof.

In another aspect the invention relates to a combination comprising an isolated strain of Epichloë endophyte that is AR128 or AR166 and a host plant.

In another aspect the invention relates to a host plant infected with an isolated strain of Epichloë endophyte that is AR128 or AR166.

In another aspect the invention relates to a plant seed infected with an isolated strain of Epichloë endophyte that is AR128 or AR166.

In another aspect the invention relates to a method of making an artificial host plant/Epichloë endophyte combination that does not induce Epichloë toxicosis in animals upon consumption, or that induces reduced Epichloë toxicosis in animals upon consumption, the method comprising artificially infecting a host plant with AR128 or AR166.

In another aspect the invention relates to a method of conferring at least some level of pest protection on a host plant comprising artificially infecting the host plant with AR128 or AR166 to form a host plant/Epichloë endophyte combination.

In another aspect the invention relates to a method of reducing the average level of Epichloë toxicosis experienced by an animal consuming an Epichloë infected host plant comprising feeding the animal with a host plant that is artificially infected with AR128 or AR166, wherein the reduction in the average level of Epichloë toxicosis experienced by the animal is an average reduction relative to the average level of Epichloë toxicosis experienced by the same type of animal consuming a host plant that is infected with a selected or wild-type Epichloë endophyte.

In another aspect the invention relates to a method of reducing the average level of Epichloë toxicosis experienced by an animal consuming an Epichloë infected host plant, the method comprising planting an area of land with a host plant or with the seed of a host plant that has been artificially infected with AR128 or AR166, and then feeding the animal with host plant material from the area of land and/or feeding the animal by grazing the animal on the area of land, wherein the reduction in the average level of Epichloë toxicosis experienced by the animal is an average reduction relative to the average level of Epichloë toxicosis experienced by the same type of animal consuming a host plant that is infected with a selected or wild-type Epichloë endophyte.

In another aspect the invention relates to a method of increasing the yield of a livestock animal comprising artificially infecting a host plant with AR128 or AR166 to form a host plant/Epichloë endophyte combination, and feeding the combination to an animal, wherein consumption of the combination by the animal results in reduced Epichloë toxicosis as compared to consumption of another host plant/epoxyjanthitrem producing Epichloë endophyte combination.

Various embodiments of the different aspects of the invention as discussed above are also set out below in the detailed description of the invention, but the invention is not limited thereto. Other aspects of the invention may become apparent from the following description that is given by way of example only and with reference to the accompanying drawings.

### BRIEF DESCRIPTION OF THE DRAWINGS

The invention will now be described by way of example only and with reference to the drawings in which:

### Figure Captions

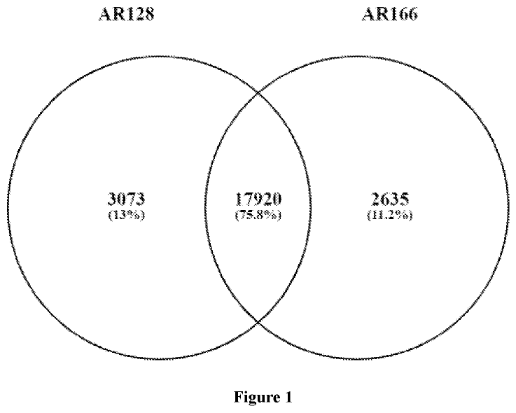

FIG. 1. Venn diagram of unique and overlapping AR128 and AR166 SNPs using the AR37 genome as a reference.

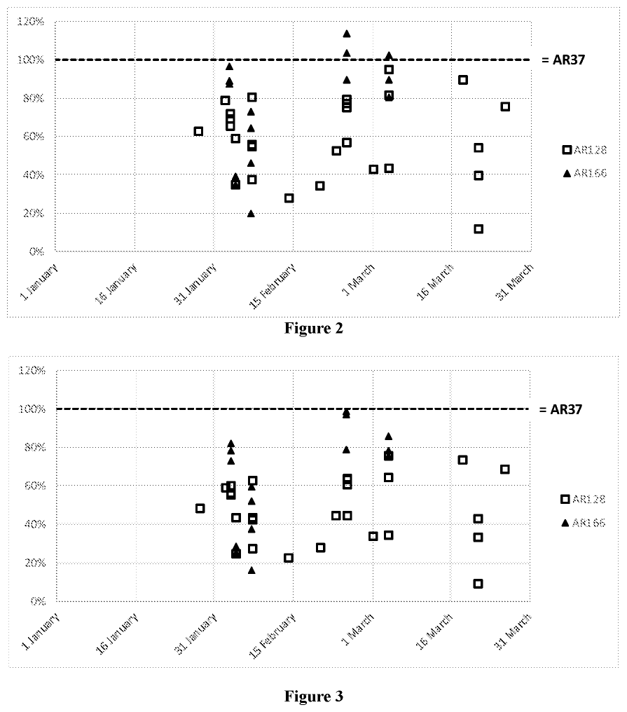

FIG. 2. Total epoxyjanthitrems in AR128 and AR166 during the months of January to March relative to AR37 (AR37=100% at all sampling dates).

FIG. 3. Epoxyjanthitrem I in AR128 and AR166 during the months of January to March relative to AR37 (AR37=100% at all sampling dates).

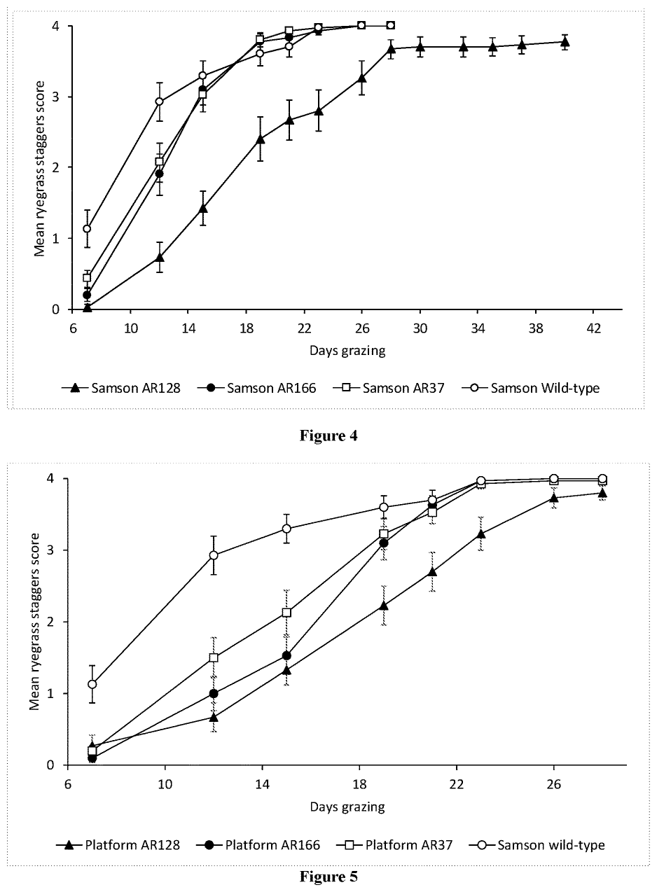

FIG. 4. Mean ryegrass staggers scores of lambs grazing Samson treatments from eight scoring dates for wild-type, AR37 and AR166, and 13 scoring dates for AR128. Error bars indicate standard error of the mean.

FIG. 5. Mean ryegrass staggers scores of lambs grazing the Platform treatments AR128, AR166 and AR37, compared with Samson wild-type, from eight scoring dates over a 28-day duration. Error bars indicate standard error of the mean.

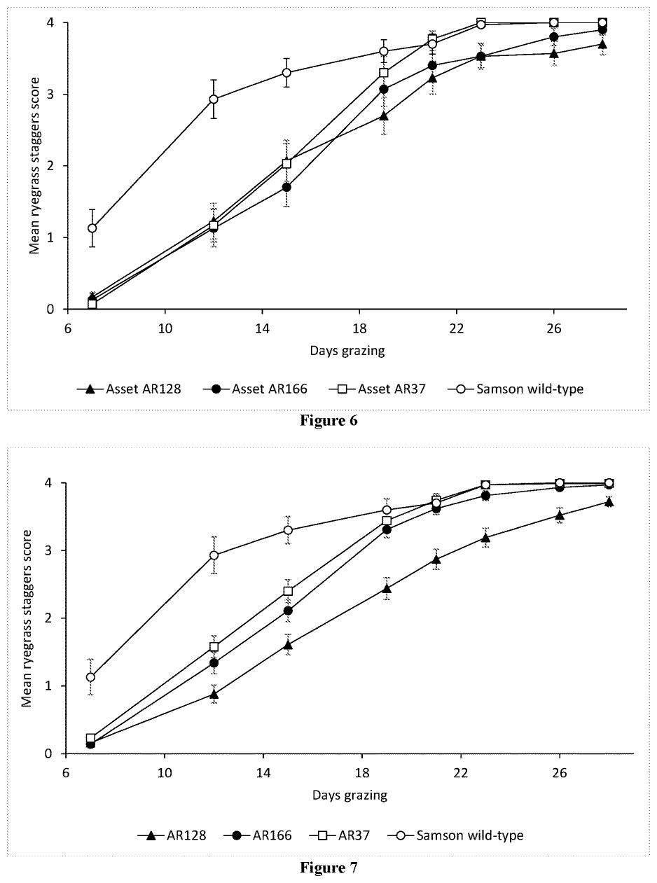

FIG. 6. Mean ryegrass staggers scores of lambs grazing the Asset treatments AR128, AR166 and AR37, compared with Samson wild-type, from eight scoring dates over a 28-day duration. Error bars indicate standard error of the mean.

FIG. 7. Mean ryegrass staggers score of lambs grazing AR128, AR166 and AR37 endophytes combined across cultivars, compared with Samson wild-type, from eight scoring dates over a 28-day duration. Error bars indicate standard error of the mean.

### DETAILED DESCRIPTION OF THE INVENTION

### Definitions

The following definitions are presented to better define the present invention and as a guide for those of ordinary skill in the art in the practice of the present invention.

Unless otherwise specified, all technical and scientific terms used herein are to be understood as having the same meanings as is understood by one of ordinary skill in the relevant art to which this disclosure pertains. Examples of definitions of common terms in botany, microbiology, molecular biology and biochemistry can be found in Biology of Plants, Raven et al. (eds.), W.H. Freeman and Company, (2005); Plant Physiology, Taiz et al. (eds.), Sinauer Associates, Incorporated, (2010); Botany: An Introduction to Plant Biology, J. D. Mauseth, Jones & Bartlett Learning, (2003); Methods for General and Molecular Microbiology, 3rd Edition, C. A. Reddy et al. (eds.), ASM Press, (2008); Encyclopedia of Microbiology, 2nd ed., Joshua Lederburg, (ed.), Academic Press, (2000); Microbiology By Cliffs Notes, I. Edward Alcamo, Wiley, (1996); Dictionary of Microbiology and Molecular Biology, Singleton et al. (2d ed.) (1994); Biology of Microorganisms 11^{th} ed., Brock et al., Pearson Prentice Hall, (2006); Biodiversity of Fungi: Inventory and Monitoring Methods, Mueller et al., Academic Press, (2004); Genes IX, Benjamin Lewin, Jones & Bartlett Publishing, (2007); The Encyclopedia of Molecular Biology, Kendrew et al. (eds.), Blackwell Science Ltd., (1994); Molecular Biology and Biotechnology: a Comprehensive Desk Reference, Robert A. Meyers (ed.), VCH Publishers, Inc., (1995); Symbioses of grasses with seedborne fungal endophytes. Schardl C L et al. (2004) Annual Review of Plant Biology 55:315-340; and Chemotype diversity ofEpichloë, fungal symbionts of grasses, Schardl C L, Young C A, Faulkner J R, Florea S, Pan J (2012) Fungal Ecology 331-344 (Schardl et al., 2012).

It is also believed that practice of the present invention can be performed using standard botanical, microbiological, molecular biology and biochemistry protocols and procedures as known in the art, and as described, for example in Methods of Studying Root Systems, vol. 33, Wolfgang Böhm, Springer-Verlag, (1979); Root methods: A Handbook, Albert L. Smit Springer, (2000); Biodiversity of Fungi: Inventory and Monitoring Methods, Mueller et al., Academic Press, (2004); Environmental Microbiology: Methods and Protocols, J. F. T. Spencer et al., Humana Press, (2004); Environmental Microbiology, P. D. Sharma, Alpha Science International, (2005); Environmental Microbiology, J. R. Leadbetter, Gulf Professional Publishing, (2005), Molecular Cloning: A Laboratory Manual, Maniatis et al., Cold Spring Harbor Laboratory Press, (1982); Molecular Cloning: A Laboratory Manual (2 ed.), Sambrook et al., Cold Spring Harbor Laboratory Press, (1989); Guide to Molecular Cloning Techniques Vol. 152, S. L. Berger and A. R. Kimmerl (Eds.), Academic Press Inc., (1987); Biotechnology of Endophytic Fungi of Grasses. 1994 Bacon and White (Eds.), and other commonly available reference materials relevant in the art to which this disclosure pertains, and which are all incorporated by reference herein in their entireties.

The term “plant” as used herein encompasses whole plants and all parts of a plant from all stages of a plant life cycle including but not limited to vegetative and reproductive cells and tissues, propagules, seeds, embryos, shoots, stems, leaves, leaf sheaths and blades, inflorescences, roots, anthers, ligules, palisade, mesophyll, epidermis, auricles, palea, lemma and tillers.

The term, “Epichloë” as used herein refers to Epichloë, a genus of endophytic fungi comprising fungal endophytes from two previously named genera; the members of the anamorphic form genus Neotyphodium and the members of the teleomorphic genus Epichloë (Leuchtmann et al., 2014).

The term, “Epichloë endophyte” as used herein refers to an endophyte of the genus Epichloë that is known in the art, or that has been shown herein, to form a symbiotic association with a host plant.

Total epoxyjanthitrem (EJ) compounds as used herein refers to the combined total of the five epoxyjanthitrems produced in planta by AR37, AR128 or AR166. These five epoxyjanthitrems are epoxyjanthitriol (EJ triol), epoxyjanthitrem I (EJ I), epoxyjanthitrem II (EJ II), epoxyjanthitrem III (EJ III), and epoxyjanthitrem IV (EJ IV), as described in Finch et al., 2020.

The abbreviation “ppm” as used herein means ‘parts per million’ dry weight of the host plant infected with the endophyte, e.g., μg per g.

The terms “Epichloë toxicosis” and “Epichloë alkaloid toxicosis” and grammatical variations thereof as used herein refer to Epichloë endophyte-derived alkaloid toxicosis which can result in reductions in animal productivity (e.g. weight gain and milk production), a reduced ability to regulate body temperature particularly when under heat stress, impaired neurology (“ryegrass staggers”) and increased faecal soiling of the breech area of sheep (“dags”) leading to higher incidence of myiasis (“flystrike”). In one embodiment, Epichloë toxicosis is ryegrass staggers.

The terms, “artificially infecting” and “artificial inoculation” as used herein encompass any inoculation of a plant, particularly a plant, preferably a grass plant, preferably a Lolium spp. (ryegrasses) plant, preferably L. perenne, L. boucheanum, and/or L. multiflorum, with AR128 or AR166 to form a plant/fungal symbiotic association that is not known from nature.

The term “in planta” as used herein in the context of fungal endophytes means a combination of an isolated strain of Epichloë endophyte AR128 or AR166 and a host plant, wherein the endophyte is living symbiotically within the host plant.

The terms “ans014”, “ans015”, “ans016”, “ans017”, “ans019”, “ans024”, “ans025”, “ans030”, “ans031”, “ans033”, “ans036”, “ans044”, “ans047”, “ans049”, “ans054”, “ans056”, “egs002”, “egs004”, “egs0010”, “egs027”, “B10” and “B11” as used herein specifically refer to the alleles having these labels as shown in Table 3.

Feeding encompasses providing harvested plant/endophyte combination material to an animal as well as grazing an animal on an area of land comprising the plant/endophyte combination.

The term “statistically significant” as used herein refers to the likelihood that a result or relationship is caused by something other than random chance. A result may be found to be statistically significant using statistical hypothesis testing as known and used in the art. Statistical hypothesis testing provides a “P-value” as known in the art, which represents the probability that the measured result is due to random chance alone. It is believed to be generally accepted in the art that levels of significance of 5% (0.05) or lower are considered to be statistically significant.

The term “comprising” as used in this specification means “consisting at least in part of”. When interpreting statements in this specification that include that term, the features, prefaced by that term in each statement, all need to be present but other features can also be present. Related terms such as “comprise” and “comprised” are to be interpreted in the same manner.

The term “consisting essentially of” as used herein means the specified materials or steps and those that do not materially affect the basic and novel characteristic(s) of the claimed invention.

The term “consisting of” as used herein means the specified materials or steps of the claimed invention, excluding any element, step, or ingredient not specified in the claim.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### Detailed Description

Many cool-season grasses (Poaceae, subfam. Pooideae) possess seed transmitted Epichloë fungal endophytes that are known for their bioprotective properties, and especially for production of anti-pest alkaloids such as lolines (Zhang et al., 2010) and peramine (Koulman et al., 2007). Asexual Epichloë (previously termed Neotyphodium species) are primarily or entirely transmitted vertically, whereas the sexual structures (stromata) of other related Epichloë species can give rise to horizontally transmissible spores (ascospores) (Zhang et al., 2010).

Symbiotic associations between Epichloë fungi and host grasses are common, and molecular phylogenetic evidence suggests that the species specificity observed in these symbiotic associations is due to the co-evolution of these groups of plants and fungal endophytes (Schardl et al., 2008).

Generally speaking, symbiotic associations formed between host plants and their Epichloë fungal endophytes are based on complex and intimate biological interactions which lead to a high degree of species specificity for both the endophyte and host (Simpson and Mace, 2012).

Additionally, as a result of a lengthy research program, the applicants have identified Epichloë endophytes that produce in combination with a host plant (i.e., “in planta”), levels of epoxyjanthitrem compounds that confer pest protection on the host plant, as compared to an uninfected control plant, while reducing the levels of Epichloë toxicosis suffered by animals feeding on the combination.

Accordingly, in one aspect, the present invention relates to an isolated strain of Epichloë endophyte wherein the endophyte comprises an ans017 allele size of 306±0.8 base pairs (bp), an ans030 allele size of 308±0.8 bp, an ans033 allele size of 171±0.8 bp, an ans054 allele size of 297±0.8 bp, an egs002 allele size of 297±0.8 bp and an egs027 allele size of 344±0.8 bp.

In one embodiment the isolated strain of Epichloë endophyte produces in planta about 2 ppm to about 60 ppm of epoxyjanthitrem compounds.

In one embodiment the about 2 ppm to about 60 ppm of epoxyjanthitrem compounds is produced in planta during austral summer. In one embodiment austral summer is January to March.

In one embodiment the isolated strain of Epichloë endophyte does not produce, in planta, more than about 0.1 ppm ergovaline or more than about 0.1 ppm lolitrem B or both.

In another aspect, the invention relates to an isolated strain of Epichloë endophyte that is AR128 (NRRL 68010) or AR166 (NRRL 68011) or a combination thereof.

Epichloë endophyte strains described herein were isolated from perennial ryegrass collected in Italy and were deposited at The United States Department of Agriculture, Agricultural Research Service Midwest Area, National Center for Agricultural Utilization Research, 1815 North University Street, Peoria, Illinois, 61604-3902, USA on the following dates for strains:
AR128 (NRRL 68010) on 23 Dec. 2020,
AR166 (NRRL 68011) on 23 Dec. 2020,
according to the Budapest Treaty for purposes of patent procedure.

Epichloë endophytes strains as described herein were isolated from endophyte-infected plants following surface sterilisation of plant tissue as described by Christensen et al. (2002).

Once isolated, the isolated and/or biologically pure fungal endophyte may be cultured using standard techniques as known in the art and as disclosed herein, including in the examples.

In one embodiment, AR128 or AR166 is cultured on antibiotic potato dextrose agar (ABPDA) between 20° C. and 25° C., preferably between 21° C. and 23° C. The optimal temperature for growth of the fungal endophyte is 22° C. Growth of the fungal endophyte at temperatures above or below this range may be possible although growth may be reduced or may cease entirely. In one embodiment, the fungal endophyte is cultured in the dark.

In one embodiment, AR128 or AR166 comprises an ans017 allele size of 306±0.8 base pairs (bp), an ans030 allele size of 308±0.8 bp, an ans033 allele size of 171±0.8 bp, an ans054 allele size of 297±0.8 bp, an egs002 allele size of 297±0.8 bp and an egs027 allele size of 344±0.8 bp.

In one embodiment the isolated strain of Epichloë endophyte comprises at least one additional SSR allele selected from the group of SSR markers consisting of ans014, ans015, ans016, ans019, ans024, ans025, ans031, ans032, ans035, ans036, ans044, ans047, ans049, ans056, egs004 and egs010, wherein at least one additional SSR allele has the number of base pairs (bp) as shown in Table 3, ±0.8 bp.

In one embodiment the isolated strain of Epichloë endophyte comprises at least two additional SSR alleles, preferably at least 3, at least 4, at least 5, at least 6, at least 7, at least 8, at least 9, at least 10, at least 11, at least 12, at least 13, at least 14, at least 15, at least 16, at least 17, at least 18 additional SSR alleles, preferably 19 additional SSR alleles, wherein each of the additional SSR alleles has the number of base pairs (bp) as shown in Table 3, ±0.8 bp.

In one embodiment the isolated strain of Epichloë endophyte comprises alleles from the following 24 SSR marker loci: B10, B11, ans014, ans015, ans016, ans017, ans019, ans024, ans025, ans030, ans031, ans032, ans033, ans035, ans036, ans044, ans047, ans049, ans054, ans056, egs002, egs004, egs010 and egs027 wherein the 24 SSR alleles have the number of base pairs (bp) as shown in Table 3, ±0.8 bp.

In one embodiment the isolated strain of Epichloë endophyte produces in planta about 2 ppm to about 60 ppm epoxyjanthitrem compounds, or less.

In one embodiment the about 2 ppm to about 60 ppm of epoxyjanthitrem compounds is produced in planta during January to March in the Southern Hemisphere and/or during July to September in the Northern Hemisphere.

In one embodiment the isolated strain of Epichloë endophyte does not produce, in planta, more than about 0.1 ppm ergovaline or more than about 0.1 ppm lolitrem B or both.

In another aspect the invention relates to a combination comprising an isolated strain of Epichloë endophyte wherein the endophyte comprises an ans017 allele size of 306±0.8 base pairs (bp), an ans030 allele size of 308±0.8 bp, an ans033 allele size of 171±0.8 bp, an ans054 allele size of 297±0.8 bp, an egs002 allele size of 297±0.8 bp and an egs027 allele size of 344±0.8 bp.

In another aspect the invention relates to a combination comprising an isolated strain of Epichloë endophyte that is AR128 or AR166 and a host plant.

In one embodiment, the combination produces in planta about 2 ppm to about 60 ppm of epoxyjanthitrem compounds.

In one embodiment the about 2 ppm to about 60 ppm of epoxyjanthitrem compounds are produced in planta during January to March in the Southern Hemisphere and/or during July to September in the Northern Hemisphere.

In one embodiment the combination produces in planta about 10% less, preferably about 15% less, about 20% less, preferably about 25% less total epoxyjanthitrem compounds than another epoxyjanthitrem producing endophyte.

In one embodiment the combination produces in planta 10% less, preferably 15% less, 20% less, preferably 25% less total epoxyjanthitrem compounds than another epoxyjanthitrem producing endophyte.

In one embodiment the combination produces about 30% less, preferably about 35% less, preferably about 40% less total epoxyjanthitrem compounds than another epoxyjanthitrem producing endophyte.

In one embodiment the combination produces 30% less, preferably 35% less, preferably 40% less total epoxyjanthitrem compounds than another epoxyjanthitrem producing endophyte.

In one embodiment the combination in planta produces about 20% to about 50% less total epoxyjanthitrem compounds, preferably about 25% to about 45% less total epoxyjanthitrem compounds than another epoxyjanthitrem producing endophyte.

In one embodiment the combination in planta produces 20% to 50% less total epoxyjanthitrem compounds, preferably 25% to 45% less total epoxyjanthitrem compounds than another epoxyjanthitrem producing endophyte.

In one embodiment the endophyte is AR128 and the combination in planta produces about 35% to about 45% less, preferably about 40% less total epoxyjanthitrem compounds than another epoxyjanthitrem producing endophyte.

In one embodiment the endophyte is AR128 and the combination in planta produces 35% to 45% less, preferably 40% less total epoxyjanthitrem compounds than another epoxyjanthitrem producing endophyte.

In one embodiment the endophyte is AR166 and the combination in planta produces about 20% to about 30% less, preferably about 25% less total epoxyjanthitrem compounds than another epoxyjanthitrem producing endophyte.

In one embodiment the endophyte is AR166 and the combination in planta produces 20% to 30% less, preferably 25% less total epoxyjanthitrem compounds than another epoxyjanthitrem producing endophyte.

In one embodiment the another epoxyjanthitrem producing endophyte is AR37.

In one embodiment the production in planta of less total epoxyjanthitrem compounds occurs during January to March in the Southern Hemisphere and/or during July to September in the Northern Hemisphere.

In one embodiment the combination produces in planta about 20% less epoxyjanthitrem I, preferably about 25% less, about 30% less, about 35% less, preferably about 36% less than another epoxyjanthitrem I producing endophyte.

In one embodiment the combination produces in planta 20% less epoxyjanthitrem I, preferably 25% less, 30% less, 35% less, preferably 36% less than another epoxyjanthitrem I producing endophyte.

In one embodiment the combination produces in planta about 45% less epoxyjanthitrem I, preferably about 50% less, preferably about 52% less epoxyjanthitrem I than another epoxyjanthitrem I producing endophyte.

In one embodiment the combination produces in planta 45% less epoxyjanthitrem I, preferably 50% less, preferably 52% less epoxyjanthitrem I than another epoxyjanthitrem I producing endophyte.

In one embodiment the combination in planta produces about 35% to about 55% less epoxyjanthitrem I, preferably about 36% to about 52% less epoxyjanthitrem I than another epoxyjanthitrem producing endophyte.

In one embodiment the combination in planta produces 35% to 55% less epoxyjanthitrem I, preferably 36% to 52% less epoxyjanthitrem I than another epoxyjanthitrem producing endophyte.

In one embodiment the endophyte is AR128 and the combination in planta produces about 45% to about 55% less, preferably about 50% to about 53% less, preferably about 52% less epoxyjanthitrem I than another epoxyjanthitrem producing endophyte.

In one embodiment the endophyte is AR128 and the combination in planta produces 45% to 55% less, preferably 50% to 53% less, preferably 52% less epoxyjanthitrem I than another epoxyjanthitrem producing endophyte.

In one embodiment the endophyte is AR166 and the combination in planta produces about 30% to about 40% less, preferably about 35% to about 35%, preferably about 36% less epoxyjanthitrem I than another epoxyjanthitrem producing endophyte.

In one embodiment the endophyte is AR166 and the combination in planta produces 30% to 40% less, preferably 35% to 35%, preferably 36% less epoxyjanthitrem I than another epoxyjanthitrem producing endophyte.

In one embodiment the another epoxyjanthitrem I producing endophyte is AR37.

In one embodiment the production in planta of less epoxyjanthitrem I occurs during January to March in the Southern Hemisphere and/or during July to September in the Northern Hemisphere.

In one embodiment the host plant is a grass plant or part thereof, preferably a Lolium spp. plant, preferably L. perenne, L. boucheanum, or L. multiflorum, or a cultivar thereof, preferably L. perenne cultivar Grasslands Samson, preferably L. boucheanum cultivar Platform or preferably L. multiflorum cultivar Asset.

In one embodiment, the host plant is a Festuca spp plant. In one embodiment the Festuca spp is Festuca arundinacea.

In one embodiment the host plant is a Schedonorus spp plant. In one embodiment, the Schedonorus spp is Schedonorus arundinaceus.

In one embodiment the part thereof of the host plant is a plant cell line or plant callus.

In one embodiment the combination produces insufficient alkaloids in planta to cause Epichloë alkaloid toxicosis in an animal that feeds on the combination.

In one embodiment the combination produces a level of alkaloids in planta that causes reduced Epichloë toxicosis in an animal feeding on the combination as compared to the same type of animal feeding on another epoxyjanthitrem producing Epichloë endophyte/host plant combination.

In one embodiment, the level of Epichloë toxicosis induced the animal when feeding on the combination is reduced during January to March in the Southern Hemisphere and/or during July to September in the Northern Hemisphere as compared to the level of Epichloë toxicosis induced in the same type of animal feeding on other epoxyjanthitrem producing Epichloë endophyte/host plant combinations during the same time frame.

In one embodiment the animal is a grazing animal.

Specifically contemplated as embodiments of the aspects of the invention related to combinations as described herein are all of the embodiments set forth above that relate to the aspects of the invention that are isolated Epichloë endophytes as described herein.

In another aspect the invention relates to a host plant infected with an isolated strain of Epichloë endophyte wherein the endophyte comprises an ans017 allele size of 306±0.8 base pairs (bp), an ans030 allele size of 308±0.8 bp, an ans033 allele size of 171±0.8 bp, an ans054 allele size of 297±0.8 bp, an egs002 allele size of 297±0.8 bp and an egs027 allele size of 344±0.8 bp.

In another aspect the invention relates to a host plant infected with an isolated strain of Epichloë endophyte that is AR128 or AR166.

Specifically contemplated as embodiments of the aspects of the invention related to host plants infected with an isolated strain of Epichloë endophyte as described herein are all of the embodiments set forth above that relate to the aspects of the invention that are combinations comprising an Epichloë endophyte as described herein and a host plant.

In another aspect the invention relates to a plant seed infected with an isolated strain of Epichloë endophyte wherein the endophyte comprises an ans017 allele size of 306±0.8 base pairs (bp), an ans030 allele size of 308±0.8 bp, an ans033 allele size of 171±0.8 bp, an ans054 allele size of 297±0.8 bp, an egs002 allele size of 297±0.8 bp and an egs027 allele size of 344±0.8 bp.

In another aspect the invention relates to a plant seed infected with an isolated strain of Epichloë endophyte that is AR128 or AR166.

In one embodiment the plant seed has been artificially infected with the Epichloë endophyte.

In one embodiment the plant seed is a seed of a Lolium species.

In one embodiment the Lolium spp. is Lolium perenne or a cultivar thereof, preferably L. perenne cultivar Grasslands Samson (subsequently referred to as ‘Samson’).

In one embodiment the Lolium spp. is Lolium boucheanum (syn. Lolium x hybridum) or a cultivar thereof, preferably L. boucheanum cultivar Platform.

In one embodiment the Lolium spp. is Lolium multiflorum, preferably L. multiflorum cultivar Asset.

In one embodiment the Lolium spp. is Lolium arundinaceum.

In one embodiment, the plant seed is a seed of a Festuca species. In one embodiment the Festuca spp is Festuca arundinacea.

In one embodiment the plant seed is a seed of a Schedonorus species. In one embodiment, the Schedonorus species is Schedonorus arundinaceus.

Specifically contemplated as embodiments of the aspects of the invention related to plant seeds as described herein are all of the embodiments set forth above that relate to the aspects of the invention that are combinations comprising an Epichloë endophyte as described herein and a host plant and host plants comprising an Epichloë endophyte as described herein.

In another aspect the invention relates to a method of making an artificial host plant/Epichloë endophyte combination that does not induce Epichloë toxicosis in animals upon consumption, or that induces minimal Epichloë toxicosis in animals upon consumption, the method comprising artificially infecting a host plant with AR128 or AR166.

In some embodiments, artificial inoculation into a host plant may be carried out using seedlings that have been germinated for about two weeks. Preferably the seedlings have been germinated for 4 to 9 days.

Outside of this range, seedlings may still form effective associations but in some cases may be too young or too old for establishment of a mutualistic association. Seeds need to be free of non-target fungi and bacteria to ensure that the seedlings are not overcome by microbial contamination.

In one embodiment, artificial inoculation may be carried out using basal inoculation of host plant seedlings. To effectively establish the Epichloë symbiont/host plant association, inoculation of the endophyte should be made into the host plant meristem by incision of the plant and insertion of cultured fungal mycelium.

In one embodiment the combination produces, in planta, about 2 ppm to about 60 ppm total epoxyjanthitrem compounds.

In some embodiments, the method further comprises metabolic profiling of the host plant/Epichloë combination.

In one embodiment, metabolic profiling comprises determining the total amount of epoxyjanthitrem compounds produced by the combination. In one embodiment the epoxyjanthitrem compounds determined are epoxyjanthitrem I (EJ I), epoxyjanthitrem II (EJ II), epoxyjanthitrem III (EJ III), epoxyjanthitrem IV (EJ IV), and epoxyjanthitriol (EJ triol).

In one embodiment metabolic profiling comprises determining the total amount of EJI produced by the combination.

In one embodiment metabolic profiling comprises determining the absolute amount of EJ I, EJ II, EJ III, EJ IV or EJ triol, or any combination thereof produced by the combination.

Metabolic profiling of a host plant/Epichloë combination, particularly to identify alkaloids that are produced in the combination, is believed to be within the skill of those in the art in view of the disclosure of the present specification and common general knowledge.

In some embodiments, the method further comprises selecting a host plant/Epichloë combination that produces about 2 ppm to about 60 ppm total epoxyjanthitrem compounds, preferably less than 60 ppm total epoxyjanthitrem compounds.

Specifically contemplated as embodiments of the aspect of the invention related to a method of making an artificial host plant/Epichloë endophyte combination that does not induce Epichloë toxicosis in animals upon consumption as described are all of the embodiments set forth above that relate to the aspects of the invention that are isolated strains of Epichloë endophyte as well as combinations, host plants and seeds comprising an isolated Epichloë endophyte as described herein.

In another aspect the invention relates to a method of conferring at least some level of pest protection on a host plant comprising artificially infecting the host plant with AR128 or AR166 to form a host plant/Epichloë endophyte combination.

In one embodiment the host plant/Epichloë endophyte combination produces epoxyjanthitrem compounds epoxyjanthitrem I-IV and epoxyjanthitriol.

In one embodiment the host plant/Epichloë endophyte combination produces in planta, about 2 ppm to about 60 ppm total epoxyjanthitrem compounds.

In one embodiment the level of pest protection reduces consumption of the host plant/Epichloë endophyte combination by insect pests by at least 30%, preferably by at least 40%, 50%, 60%, 70%, 80%, 90%, 95%, 99%, 100%, 150%, 200%, 250%, preferably by at least 300% as compared to the consumption of the same species of host plant by insect pests that is not infected with the Epichloë endophyte.

In one embodiment the level of pest protection reduces consumption of the host plant/Epichloë endophyte combination by insect pests by about 30%, preferably by about 40%, 50%, 60%, 70%, 80%, 90%, 95%, 99%, 100%, 150%, 200%, 250%, preferably by at least 300% as compared to the consumption of the same species of host plant by insect pests that is not infected with the Epichloë endophyte.

In one embodiment the host plant that is not infected with the Epichloë endophyte is infected with a different Epichloë endophyte.

In one embodiment the host plant that is not infected with the Epichloë endophyte is not infected with an Epichloë endophyte.

In one embodiment, the pest is an insect pest.

In one embodiment the insect pest is selected from the group consisting of: (1) species of aphids selected from the group consisting of Rhopalosiphum padi, Schizaphis graminum, Rhopalosiphum maidis, Metopoliphium dirhodum, Sitobion spp., Sitobion avenae, Sitobion fragariae, and Diuraphis noxis; (2) species of grass and cereal flies selected from the group consisting of Oscinella frit, Oscinella pusilla, Mayetiola destructor, Cerodontha spp., Cerodontha australis, Cerodontha angustipennis, Formia fumigata, Meromyze americana, Haplodiplosis marginata, Chlorops pumilionis, Tipula spp. Chromatomyia fuscula, Cephus pygmaeus, Chromatomyia fuscula, and Contarinia tritici; (3) species of thrips selected from the group consisting of Limothrips cerealium, Limothrips denticornis, Aptinothrips rufus, and Stenothrips graminum; (4) species of grasshoppers and crickets selected from the group consisting of Locusta migratoria, Phaulacridium marginale, Phaulacridium vittatum, Melanoplus spp., and Teleogryllus commodus; (5) species of bugs Nyssius huttoni or Blissus leucopertus; (6) weevils of Sphenophorus spp.; (7) species of armyworm and cutworm selected from the group consisting of Pseudaletia unipuncta, Spodoptera spp., Mythimna separata; Persectania aversa, and Agrostis ipsilon; (8) Oulema melanopus leaf bugs; (9) species of white grubs selected from the group consisting of Popillia japonica, Costelytra zealandica, Phyllopertha spp., Rhizotrogus majalis, and Anisoplia segetum; (10) species of mealybug selected from the group consisting of Phenacoccus hordei, Balanococcus poae, Ripersella rumicis, and Porphyrophora tritici; (11) species of wireworms Conoderus spp., or Limonius spp.; (12) Zabrus tenebrioides beetles; (13) species of mites selected from the group consisting of Penthaleus spp., Halotydeus destructor, and Aceria spp.; (14) species of stored product pests selected from the group consisting of Sitophilus oryzae, Sitophilus granarius, Sitotroga cerealella, Rhyzopertha dominica, Cryptolestes spp., Oryzaephilus surinamensis, Cadra cautella, Plodia interpunctella, Tribolium confusum, Tribolium castaneum, and Lasioderma erricorne; (15) Philaenus spumarius froghoppers; (16) species of nematodes selected from the group consisting of root lesion nematodes of Pratylenchus spp. selected from the group consisting of P. thornei, P. crenatus, P. neglectus and P. penetrans, cereal cyst nematodes of Heterodera spp, and Punctodera spp. selected from the group consisting of H. avenae, H. latipons, H. hordecalis, H. filipjevi, H. mani, H. bifenestra, H. pakistanensis and P. punctata, root knot nematodes of Meloidogyne spp. selected from the group consisting of M. chitwoodi, M. naasi, M. artiellia, M. microtyla, M. ottersoni, M. graminicola, M. graminis, M. kikuyensis and M. spartinae, stem nematodes of Ditylenchus spp. selected from the group consisting of D. dipsicai and D. radicicola; and the seed gall nematode Anguina tritici; (17) species of slugs selected from the group consisting of Deroceras reticulatum, Arion hortensis agg, and A. subfuscus.

In one embodiment the insect pest is Argentine stem weevil (Listronotus bonariensis), black beetle (Heteronychus arator), porina (Wiseana cervinata or W. copularis) or root aphid (Aploneura lentisci).

In another aspect the invention relates to a method of reducing the average level of Epichloë toxicosis experienced by an animal consuming an Epichloë infected host plant comprising feeding the animal with a host plant that is artificially infected with an isolated strain of Epichloë endophyte wherein the endophyte comprises an ans017 allele size of 306±0.8 base pairs (bp), an ans030 allele size of 308±0.8 bp, an ans033 allele size of 171±0.8 bp, an ans054 allele size of 297±0.8 bp, an egs002 allele size of 297±0.8 bp and an egs027 allele size of 344±0.8 bp, wherein the reduction in the average level of Epichloë toxicosis experienced by the animal is an average reduction relative to the average level of Epichloë toxicosis experienced by the same type of animal consuming a host plant that is infected with a selected or wild-type Epichloë endophyte.

In another aspect the invention relates to a method of reducing the average level of Epichloë toxicosis experienced by an animal consuming an Epichloë infected host plant comprising feeding the animal with a host plant that is artificially infected with AR128 or AR166, wherein the reduction in the average level of Epichloë toxicosis experienced by the animal is an average reduction relative to the average level of Epichloë toxicosis experienced by the same type of animal consuming a host plant that is infected with a selected or wild-type Epichloë endophyte.

In one embodiment the selected endophyte is an epoxyjanthitrem producing Epichloë endophyte.

In one embodiment the selected endophyte is AR37.

In one embodiment the host plant is a Lolium spp. plant.

In one embodiment the Lolium spp. is Lolium perenne or a cultivar thereof, preferably L. perenne cultivar Grasslands Samson (subsequently referred to as ‘Samson’).

In one embodiment the Lolium spp. is Lolium boucheanum (syn. Lolium x hybridum) or a cultivar thereof, preferably L. boucheanum cultivar Platform.

In one embodiment the Lolium spp. is Lolium multiflorum, preferably L. multiflorum cultivar Asset.

In one embodiment the Lolium spp. is Lolium arundinaceum.

In one embodiment, the host plant is a Festuca spp plant. In one embodiment the Festuca spp is Festuca arundinacea.

In one embodiment the host plant is a Schedonorus spp plant. In one embodiment, the Schedonorus spp is Schedonorus arundinaceus.

In another aspect the invention relates to a method of reducing the average level of Epichloë toxicosis experienced by an animal consuming an Epichloë infected host plant, the method comprising planting an area of land with a host plant or with the seed of a host plant that has been artificially infected with an isolated strain of Epichloë endophyte wherein the endophyte an ans017 allele size of 306±0.8 base pairs (bp), an ans030 allele size of 308±0.8 bp, an ans033 allele size of 171±0.8 bp, an ans054 allele size of 297±0.8 bp, an egs002 allele size of 297±0.8 bp and an egs027 allele size of 344±0.8 bp, and then feeding the animal with host plant material harvested from the area of land and/or feeding the animal by grazing the animal on the area of land, wherein the reduction in the average level of Epichloë toxicosis experienced by the animal is an average reduction relative to the average level of Epichloë toxicosis experienced by the same type of animal consuming a host plant that is infected with a selected or wild-type Epichloë endophyte.

In another aspect the invention relates to a method of reducing the average level of Epichloë toxicosis experienced by an animal consuming an Epichloë infected host plant, the method comprising planting an area of land with a host plant or with the seed of a host plant that has been artificially infected with AR128 or AR166, and then feeding the animal with host plant material harvested from the area of land and/or feeding the animal by grazing the animal on the area of land, wherein the reduction in the average level of Epichloë toxicosis experienced by the animal is an average reduction relative to the average level of Epichloë toxicosis experienced by the same type of animal consuming a host plant that is infected with a selected or wild-type Epichloë endophyte.

In one embodiment the selected endophyte is an epoxyjanthitrem producing Epichloë endophyte.

In one embodiment the selected endophyte is AR37.

In one embodiment the host plant is a Lolium spp. plant.

In one embodiment the Lolium spp. is Lolium perenne or a cultivar thereof, preferably L. perenne cultivar Grasslands Samson (subsequently referred to as ‘Samson’).

In one embodiment the Lolium spp. is Lolium boucheanum (syn. Lolium x hybridum) or a cultivar thereof, preferably L. boucheanum cultivar Platform.

In one embodiment the Lolium spp. is Lolium multiflorum, preferably L. multiflorum cultivar Asset.

In one embodiment the Lolium spp. is Lolium arundinaceum.

In one embodiment, the host plant is a Festuca spp plant. In one embodiment the Festuca spp is Festuca arundinacea.

In one embodiment the host plant is a Schedonorus spp plant. In one embodiment, the Schedonorus spp is Schedonorus arundinaceus.

In one embodiment feeding comprises feeding while deterring herbivory by insect pests.

In one embodiment feeding comprises feeding in austral summer. In one embodiment, austral summer is January to March.

In one embodiment the area of land is a pre-determined area of land on which deterrence or reduction of pest damage is desired. In one embodiment the area of land, or pre-determined area of land is a verge, divider, clearing, field, meadow, pasture or paddock.

In one embodiment the area of land is used in agriculture.

In one embodiment, at least 10%, preferably at least 20%, at least 30%, at least 40%, at least 50%, at least 60%, at least 70%, at least 80%, at least 90%, at least 95%, preferably about 99%, preferably all of the area of land is planted.

In one embodiment, at least 10%, preferably about 20%, 30%, 40%, 50%, 60%, 70%, 80%, 90%, 95%, preferably about 99%, preferably all of the area of land is planted.

In one embodiment the level of pest protection reduces consumption of the host plant/Epichloë endophyte combination by insect pests by at least 30%, preferably by at least 40%, 50%, 60%, 70%, 80%, 90%, 95%, 99%, 100%, 150%, 200%, 250%, preferably by at least 300% as compared to an area of land that is the same size, but that has not been planted with a host plant infected with the Epichloë endophyte, the combination or with infected plant seed.

In one embodiment the level of pest protection reduces consumption of the host plant/Epichloë endophyte combination by insect pests by about 30%, preferably by about 40%, 50%, 60%, 70%, 80%, 90%, 95%, 99%, 100%, 150%, 200%, 250%, preferably by at least 300% as compared to an area of land that is the same size, but that has not been planted with a host plant infected with the Epichloë endophyte, the combination or with infected plant seed.

In another aspect the invention relates to a method of increasing the yield of a livestock animal comprising artificially infecting a host plant with AR128 or AR166 to form a host plant/Epichloë endophyte combination, and feeding the combination to an animal, wherein consumption of the combination by the animal results in reduced Epichloë toxicosis as compared to consumption of another host plant/epoxyjanthitrem producing Epichloë endophyte combination.

Specifically contemplated as embodiments of this aspect of the invention are all of the embodiments related to the other method aspects of the invention, including embodiments related to Epichloë endophytes comprising defined allele sizes, AR128 and/or AR166, host plants, seasonal variations in alkaloid levels and/or insect resistance, and other Epichloë endophytes, particularly epoxyjanthitrem producing endophytes.

Various aspects of the invention will now be illustrated in non-limiting ways by reference to the following examples.

### EXAMPLES

### Example 1

### Detection of Fungal Endophyte Strains

Source and geographic origin of AR128 and AR166.

Table 1 shows the source accession number from which the isolated strains of Epichloë endophyte as described herein were obtained, the species of the natural host plant accession, and the regional source of the accession.

TABLE 1
Strains of isolated endophytes by AR code number, original host
species, regional source, and source accession number.

| Endophyte | Host grass | Region | Source accession |
|---|---|---|---|
| AR128 | Lolium perenne | Italy | 56085 |
| AR166 | Lolium perenne | Italy | 61701 |

The endophytes in this invention were identified from wild ryegrass plants collected from field sites in Italy. The collected plants were assessed for the presence of Epichloë endophyte using the immuno-detection method of Simpson et al. (2012). Seeds from each Epichloë endophyte-infected plant were harvested and despatched to New Zealand for further examination.

As detailed further herein, selecting the inventive strains AR128 and AR166 detailed herein required the screening of >3000 seed accessions from around the globe. Current methodologies only allow for the screening of 30-40 accessions in one experiment, taking 3-4 months to complete the analyses required to determine the alkaloids produced and the genetic distinctiveness from already catalogued strains. Our current collection contains 188 characterized strains, of which there are only four unique epoxyjanthitrem-producing strains. For the epoxyjanthitrem-producing strains, a further 6-9 months experimentation was required to determine the epoxyjanthitrem profiles and compare relative levels of production in a ryegrass host. Of the four epoxyjanthitrem-producing strains, only AR128 and AR166 showed the alkaloid profile described therein.

### Example 2

### Detection of Genetic Variation of Fungal Endophyte Strains

Endophyte strains AR128 and AR166 were characterised and distinguished for genetic variation by DNA ‘fingerprinting’ based on genotypic data derived from up to 24 selected simple sequence repeat (SSR) marker loci using primer sequences of Table 2. These primer sequences had previously been shown to generally amplify Epichloë endophyte polymorphic DNA sequences from when the endophytes are in planta (Moon et al., 1999; Kirkby et al., 2011; Simpson et al., 2012; Card et al., 2014.)

Samples of about 100 mg fresh weight of basal tiller were used to extract total genomic DNA (plant+endophyte), following the plant DNA isolation procedure of the FastDNA kit as recommended by the manufacturer (MP Biomedicals, Solon, Ohio, USA) for plant samples.

SSR amplification was conducted with oligonucleotide primer pairs, using one of two polymerase chain reaction (PCR) protocols (Table 2). In both protocols, PCR was carried out using an iCycler thermocycler (BioRad, Hercules, California, USA).

Protocol 1 was as described by Moon (Moon et al., 1999), except that an annealing temperature of 60° C. was used. In this protocol forward primers were labelled at the 5′ terminus with the fluorophore 6-FAM™ (Applied Biosystems, Foster City, California, USA).

In Protocol 2 forward primers were synthesised with a 21 nucleotide M13 tail sequence at the 5′-terminus (5′-TGTAAAACGACGGCCAGT-3′) (SEQ ID NO: 1), to facilitate universal labelling of PCR products by a 6-FAM™-labelled M13 primer (Schuelke, 2000). Reverse primers were synthesised with the sequence 5′-GTTTCTT-3′ (SEQ ID NO: 2) at the 5′-terminus end to promote non-templated adenylation at the 3′-terminus end of PCR product (Brownstein et al., 1996). A 10 μL PCR reaction volume was used, containing approximately 10 ng of total genomic DNA, 2.5 mM magnesium chloride, 1×PCR buffer, 0.05 mM of each dNTP, 0.0375 μM forward primer, 0.15 μM reverse primer, 0.15 μM of fluorescent-labelled M13 primer and 0.75 U of Platinum Taq DNA polymerase (Invitrogen, Carlsbad, California). PCR was carried out using the following profile: (1) 94° C. for 4:00 minutes, (2) 30 cycles of: 94° C. for 30 seconds, 55° C. for 30 seconds and 72° C. for 30 seconds, (3) 8 cycles of: 94° C. for 30 seconds, 53° C. for 30 seconds and 72° C. for 30 seconds, (4) 72° C. for 30 minutes (after Schuelke, 2000).

PCR products were analysed by capillary electrophoresis on an ABI 3130xl Genetic Analyser using a 22 cm capillary array with POP-7™ polymer (Applied Biosystems). GS500 LIZ (Applied Biosystems) was used as an internal size standard. Electropherograms were analysed using ABI Prism GeneScan (v 3.7, Applied Biosystems), and genotype data was scored using Genemarker analysis software (SoftGenetics LLC, Pennsylvania, USA).

The inventors note here that in their experience, allele sizes will vary in some analyses according to a number of factors. For example, estimates of fragment (allele) sizes based on capillary electrophoresis are affected by factors including, but not limited to, the type of instrument, the length of the capillary array, the type of polymer used and environmental variables including ambient temperature. Accordingly, the SSR allele sizes in bp that are reported herein, including those in Table 3, are associated with the analysis platform described and also include a confidence interval of ∞ 0.8 bp.

Plants examined above were then further characterised by performing chemical analyses. Six infected seedlings were further examined for the presence of alkaloids, attributable to the presence of endophytes, such as indole diterpenes, ergot alkaloids, peramine and lolines.

TABLE 2
SSR primer sequences. All primer pairs used with PCR protocol 2 have additional
sequences appended to the 5′ terminus of the primer sequence: forward primers
(5′-TGTAAAACGACGGCCAGT-3′) (SEQ ID NO: 1); reverse primers 5′-GTTTCTT-3′
(SEQ ID NO: 2).

| | | | PCR | |
|---|---|---|---|---|
| SSR | Primer sequences (5′-3′) | | protocol | SEQ ID NO |
| B10 | forward | CGCTCAGGGCTACATACA | 1 | 3 |
| | | CCATGG | | |
| | reverse | CTCATCGAGTAACGCAGG | | 4 |
| | | CGACG | | |
| B11 | forward | CATGGATGGACAAGAGAT | 1 | 5 |
| | | TGCACG | | |
| | reverse | TTCACTGCTACAATTCTGT | | 6 |
| | | CCAGC | | |
| ans014 | forward | CAATCATTCTGAACCAAA | 2 | 7 |
| | | ACCA | | |
| | reverse | CCTTATACGCAGGACCAA | | 8 |
| | | AGAC | | |
| ans015 | forward | GTCTAAACAGCACAGAGC | 2 | 9 |
| | | GAGA | | |
| | reverse | ACCCGAGTCAAGTTGCATT | | 10 |
| | | ATT | | |
| ans016 | forward | CACAAAGACAAACGCCAA | 2 | 11 |
| | | AAG | | |
| | reverse | GCAAAGCTCACAGACAAA | | 12 |
| | | GGTC | | |
| ans017 | forward | GCCATCCGATAACCTGTCT | 2 | 13 |
| | | CTA | | |
| | reverse | TCGTTTCGGTTCGATTAAG | | 14 |
| | | AGT | | |
| ans019 | forward | TACCTCTGCACGGTGTATT | 2 | 15 |
| | | CC | | |
| | reverse | TGCATAACACTCACCTTAT | | 16 |
| | | AGTCG | | |
| ans024 | forward | CTGGACTACCTCCTCCACA | 2 | 17 |
| | | ATC | | |
| | reverse | GCCCGCATTAAAGAAATA | | 18 |
| | | AGG | | |
| ans025 | forward | CAGCCCTATTTTATGTTTG | 2 | 19 |
| | | AAGG | | |
| | reverse | GCTCCTGCTTTTATTCCTG | | 20 |
| | | CTA | | |
| ans030 | forward | GCATAGGAAGTGCTGTTA | 2 | 21 |
| | | ATTTGA | | |
| | reverse | AGAGTAGAACCTGCTTGC | | 22 |
| | | GTTA | | |
| ans031 | forward | ACTCGGAGACATGAAAAC | 2 | 23 |
| | | CATC | | |
| | reverse | GACGTGCTCTGTGATGTTG | | 24 |
| | | AAT | | |
| ans032 | forward | GATTCACCTCATCTACCAT | 2 | 25 |
| | | CTACC | | |
| | reverse | GGTCGAGTTTATATCTCCG | | 26 |
| | | ATG | | |
| ans033 | forward | GCGTTGAGGAGGCTAGAT | 2 | 27 |
| | | AGAA | | |
| | reverse | TTCCAAGCTGAACAAAAG | | 28 |
| | | TCAA | | |
| ans035 | forward | CGGTGCTCTTTACCTTTTG | 2 | 29 |
| | | ACT | | |
| | reverse | CATATAAGAGGCCCATCT | | 30 |
| | | CACC | | |
| ans036 | forward | ATTTGCAGCAGAGATGAT | 2 | 31 |
| | | GTGT | | |
| | reverse | CCTGCACCGGACTGTTAGT | | 32 |
| | | AAT | | |
| ans044 | forward | TCCAGAAGTAGTCGGGAT | 2 | 33 |
| | | CAGT | | |
| | reverse | ATGAGAGGGGAATTGAAG | | 34 |
| | | ACAA | | |
| ans047 | forward | TCACCTCTCTCGACGTTGT | 2 | 35 |
| | | CTA | | |
| | reverse | TCTCGTAAAACCCAATTCC | | 36 |
| | | ATC | | |
| ans049 | forward | ATCTCGGCTCTAACAAAGT | 2 | 37 |
| | | TCG | | |
| | reverse | GCATATACGGACGTGCTA | | 38 |
| | | CCTA | | |
| ans054 | forward | CTTGTGATGAGAATCGTCA | 2 | 39 |
| | | AGC | | |
| | reverse | CCCTACCGAAACTACATC | | 40 |
| | | GTTC | | |
| ans056 | forward | AGACCATCATGTGAGCGA | 2 | 41 |
| | | CTT | | |
| | reverse | GTGATGCCCTCTATGTGTA | | 42 |
| | | TGC | | |
| egs002 | forward | TAAGGCGAGGTCAGTTAT | 2 | 43 |
| | | TAGCTT | | |
| | reverse | CTCTTTTGGGACCTACCTA | | 44 |
| | | GTCAA | | |
| egs004 | forward | TATACAGACTACGAACAC | 2 | 45 |
| | | CCTCCC | | |
| | reverse | GGATGTTACTGAATGAAA | | 46 |
| | | ACTCCC | | |
| egs010 | forward | ATTCCTTGTCCTGTTGTCT | 2 | 47 |
| | | CAAAC | | |
| | reverse | ATGGTTGCTCGAAAAGAC | | 48 |
| | | TTACTC | | |
| egs027 | forward | GATGACGTATCTTGATGCT | 2 | 49 |
| | | ACCAC | | |
| | reverse | CGTGTATAAAGTTCGGGA | | 50 |
| | | TCCTAT | | |

TABLE 3
SSR allele sizes for strains AR128 and
AR166 in base pairs (bp) ± 0.8.

| | Allele size (bp) | |
|---|---|---|
| SSR | AR128 | AR166 |
| B10 | 163 | 163 |
| B11 | 132 | 132 |
| ans014 | 315 | 315 |
| ans015 | 337 | 337 |
| ans016 | 295 | 295 |
| ans017 | 306 | 306 |
| ans019 | 198 | 198 |
| ans024 | 233 | 233 |
| ans025 | 304 | 304 |
| ans030 | 308 | 308 |
| ans031 | 307 | 307 |
| ans032 | 214 | 214 |
| ans033 | 171 | 171 |
| ans035 | 241 | 241 |
| ans036 | 267 | 267 |
| ans044 | 278 | 278 |
| ans047 | 313 | 313 |
| ans049 | 282 | 286 |
| ans054 | 297 | 297 |
| ans056 | 267 | 268 |
| egs002 | 297 | 297 |
| egs004 | 356 | 356 |
| egs010 | 355 | 355 |
| egs027 | 344 | 344 |

### Example 3

### Genome Sequence

### PacBio Library Preparation, Sequencing, Processing and Assembly

For PacBio sequencing, DNA was extracted from the AR37 sample.

From the DNA extractions, a single 20 Kb SMRTbell template library was prepared from these to the manufacturer's specifications by Macrogen Inc. (Seoul, Korea) and sequenced on 5 SMRT cells in September 2018.

The resulting 463,991 raw reads, averaging around 15,000 bases in length were processed using the open source bioinformatics software Canu release v1.5 (Koren et al., 2017) to process, trim and assemble the PacBio reads into a genome of over 33 million bases with 14 scaffolds (Table 4).

TABLE 4
Sequencing statistics for the AR37 reference genome.

| Sequence statistic | Length (bp) |
|---|---|
| Total number of sequences | 14 |
| Total number of residues | 33,154,102 |
| Average length of sequences | 2,368,150 |
| Minimum sequence length | 62,691 |
| Maximum sequence length | 9,363,371 |

### Illumina Paired-End Library Preparation and Sequencing

For Illumina sequencing, DNA was extracted from 2 samples: AR128 and AR166.

From the DNA extractions, paired-end DNA libraries were prepared from these to the manufacturer's specifications by Macrogen Inc. (Seoul, Korea) using the Illumina TruSeq Nano DNA kit with inserts c. 470 bp in size and sequenced on an Illumina Hiseq2500 with 100 bp read lengths. This produced 12,405,299 and 12,188,455 raw read pairs for AR128 and AR166 respectively, resulting in 2,481,059,800 and 2,437,691,000 base pairs. This indicated an expected coverage of over 70 reads for each base on the 33 million base pair AR37 genome.

The open source bioinformatics software trimmomatic version 1.0.6 (Bolger et al., 2014) was used to trim the resulting Illumina reads. The quality window used a minimum base quality of 30 (1 in a 1,000 probability of error) over a sliding window of 4 bases, but otherwise with default settings. Only reads with at least 35 bases remaining in both reads of each pair were kept. FastQC version 0.11.8 (Andrews 2010) was used to calculate quality statistics of the raw and trimmed reads.

### Mapping Reads to the AR37 Reference Genome

The trimmed Illumina reads were mapped to the AR37 reference genome using “mem” algorithm of bwa version 0.7.17-r1188 (Li and Durbin 2009) with default settings to generate a sequence alignment (SAM) file (Li et al., 2009). The SAM file was then converted to a binary alignment (BAM) file using the “view” and “sort” executables in samtools versions 1.8 (Li et al., 2009). The preliminary set of SNPs were identified using bcftools version 1.9 (Li 2011) assuming haploid samples using only bases with a minimum base call quality of 30 (at most, a 1 in a 1,000 probability of error) derived from reads with a minimum mapping quality of 30 (at most a 1 in a 1,000 probability of incorrect mapping), resulting in variant call format (VCF) tab-delimited text files (Danecek et al., 2011). The SNPs in these files were filtered for a minimum of 10 and maximum of 100 supporting sequencing reads covering the base of interest and a minimum allele frequency of 80% (i.e. a base different to the reference had to be seen in at least 80% of the reads covering that base) and a SNP call quality (−10 log₁₀ probability that the SNP call is incorrect) of over 225. This resulted in tens of thousands of SNPs (20,993 in AR128 and 20,555 in AR166). A Venn diagram of the 2 sets of SNPs was created (FIG. 1.), using the Venny version 2.1 website (Oliveros 2007), indicating that 3,073 SNPs were unique to AR128 and 2,635 SNPs were unique to AR166 and 17,920 SNP were in common for both AR128 and AR166 when compared to AR37.

The trimmed reads that several of the SNP were called from, were examined using the open source genome viewer IGV version 2.4 (Thorvaldsdóttir et al., 2013) and confirmed by visual assessment after checking for the absence of repetitive sequence (including repeats of a single nucleotide, known as homopolymers), and contig ends near the SNPs. These SNPs clearly differentiate AR37, AR128 and AR166.

### Example 4

### Isolation of Fungal Endophyte Strains

AR128 and AR166 were isolated from endophyte-infected ryegrass seed accessions #56085 for AR128, and #61701 for AR166 following surface disinfection of plant tissue as generally known in the art, with small amendments from Christensen et al. (2002). Ryegrass tillers were removed from plants by cutting at the base and trimming to about 5 cm before surface disinfecting. Tiller sections were surface disinfected by a quick rinse in 96% ethanol and agitating in a 4.2% sodium hypochlorite solution for one minute followed by rinsing twice in sterile tap water. Tiller sections were allowed to dry on sterile filter papers (Whatman® quantitative filter paper, ashless, Grade 41) within a sterile environment before being sectioned transversely with the aid of forceps and a scalpel. Tiller sheath rings were separated and transferred to Petri plates containing potato dextrose agar (PDA, Oxoid Limited, England) plus 0.1 g/L chloramphenicol. The Petri plates were incubated in the dark at 22-25° C. for 3-5 weeks. Cultures could be sub-cultured on the same medium or PDA without the antibiotic, chloramphenicol.

Cultures were examined for colony growth rates, colony morphology and their ability to produce conidia (asexual spores) (see Example 5).

The selection of AR128 and AR166 for further examination was based on genotype and secondary metabolite profiles.

AR128 and AR166 cultures prepared, and sometimes sub-cultured in the manner of this example, were used for testing the inoculation and possible enduring infection in seedlings of Lolium perenne (and other Lolium species) as described below.

### Example 5

### Endophyte Descriptions

Fungal colony morphology was examined as described by Card et al. (2014). In summary, endophytes were sub-cultured onto PDA and incubated for 4 weeks at 22° C. in the dark, with radial dimensions measured using a digital calliper. An average measurement was calculated from five replicate colonies per endophyte strain. Each of the replicate colonies from each strain was then examined and their morphological characteristics described with respect to the shape and colour of their colonies, and the degree of colony immersion within the agar medium. Endophyte strains were additionally sub-cultured on 4% water agar. After 4 weeks incubation, at 22° C. in the dark, hyphae were observed under a stereo (or dissecting) microscope, at a magnification of 63×, for the presence of conidia (asexual spores).

In vitro characteristics of fungal strains, when grown on PDA, were consistent with published descriptions of Epichloë festucae var. lolii, syn. Neotyphodium lolii (Christensen et al., 1993; Glenn et al., 1996), being slow to moderately slow growing. Colonies raised from the agar, white, cottony, slightly to strongly convoluted, felty, with abundant aerial hyphae. Colony reverse tan to cream at margin.

### Example 6

Inoculation of Epichloë Fungal Endophytes into Lolium perenne

Seeds of Lolium perenne, cultivar Samson, were surface sterilised and inoculated with an isolated Epichloë endophyte as described herein using methodology as described by Latch and Christensen (1985). Seeds were surface disinfected by immersion in a 50% sulphuric acid solution for 15 minutes followed by a five times rinse with tap water and immersion in a 4.2% sodium hypochlorite solution for 15 minutes followed by two rinses in sterile water. Seeds were dried in a laminar flow cabinet on sterile Whatmann filter paper before transferring to Petri plates containing 4% water agar. The seeds on plates were allowed to germinate in the dark at 22-25° C. for 4-9 days and the resulting etiolated seedlings were inoculated before being returned to the incubator for a further 7 days in the dark. Petri plates were then placed under white fluorescent lights at room temperature for at least 7 days before planting them in commercial potting mix and transferring them to a glasshouse. Plants were grown for ca. 6 weeks before identifying endophyte-infected individuals using the method of Simpson et al. (2012). Plants were further grown in the field and endophyte-infected plants were examined for phenotype in comparison with the typical uninfected plants and in particular to determine whether inflorescences and seed heads would be formed, and examine if endophyte was transmitted as viable endophyte to harvested seeds.

The same procedures were used for Lolium boucheanum cultivar Platform and Lolium multiflorum cultivar Asset.

### Example 7

### Alkaloid Expression

The Epichloë endophyte strains AR128 and AR166 both produce epoxyjanthitrems I-IV and epoxyjanthitriol, which when combined at high levels can induce Epichloë toxicosis. These two endophytes do not produce peramine or either of the two classes of alkaloids that induce Epichloë toxicosis—the lolitrem alkaloids and particularly lolitrem B which is primarily responsible for “ryegrass staggers”, or any of the ergot alkaloids including ergovaline responsible for heat stress and production losses. Accordingly, these animals consuming host plants comprising either AR128 or AR166 endophytes are not susceptible to other forms of Epichloë toxicosis as would be observed in animals consuming selected or wild-type Epichloë endophytes that do produce peramine, lolitrem B and/or ergovaline.

A range of agronomic trialling of ryegrass was conducted in which the epoxyjanthitrem production of AR128 and AR166 was compared to AR37. These trials included a wide range of ryegrass cultivars, and were conducted over several years under different conditions in field trials at secure locations around New Zealand and Australia. In New Zealand, North Island field sites were at Kerikeri in Northland, Ruakura in the Waikato and Palmerston North in the Manawatu, while the South Island field site was in Canterbury at Lincoln. The North Island sites are relatively warm in winter with moderate rates of grass growth in this season, while the South Island site is cold in winter with little growth in this season. Summers can be warm and dry at all sites, with ryegrass persistence being poorest at the two most northern sites. The site in Australia near Ballarat in the state of Victoria, has a dry climate, which can have higher temperatures during the summer months than the New Zealand sites.

The experimental layout and management of most of these trials is best described by Hume et al. (2007). The trials included: AR37, AR128 and AR166 in association with cultivar Grasslands Samson diploid perennial ryegrass (Lolium perenne); AR37, AR128 and AR166 in association with cultivar Platform long-rotation diploid hybrid ryegrass (L. boucheanum); and AR37, AR128 and AR166 in association with cultivar Asset diploid Italian ryegrass (L. multiflorum).

Samples of ryegrass were cut at ground level at 5-10 random positions from each plot and analysed for alkaloids according to published protocols (Fletcher et al., 2017).

The selected endophyte strains AR128 and AR166 on average had lower total epoxyjanthitrems and lower epoxyjanthitrem I production relative to AR37 during the months when animal health issues arise due to the potential for ryegrass staggers induced by these toxins (January through to March) (Error! Not a valid bookmark self-reference., Table, FIG. 2, FIG. 3). For total epoxyjanthitrems, overall average concentrations were lower by 40% and 25% for AR128 and AR166, respectively, relative to AR37. This effect was even greater for epoxyjanthitrem I, with overall average concentrations lower by 52% and 36% for AR128 and AR166, respectively, relative to AR37. The lower levels of total epoxyjanthitrems, and lower levels of the known tremorgen epoxyjanthitrem I, mean that the ryegrass pasture is less tremorgenic and less likely to cause animal health issues, and therefore lead to improved animal performance. Though the levels of the epoxyjanthitrems are reduced, the selected endophyte strains AR128 and AR166 are able to provide substantial protection from the key insect pasture pests (Argentine stem weevil, black beetle, root aphid, and porina).

TABLE 5
Total epoxyjanthitrem production during the months of January to March for a range of
cultivar-endophyte combinations at sites in New Zealand (NZ) and Australia (AU).

| Sampling | Sampling | | Total epoxyjanthitrems (ppm) | | |
|---|---|---|---|---|---|
| Site | Date | Cultivar | AR128 | AR166 | AR37 |
| Lincoln, NZ | Mar. 1, 2016 | Samson | 2.7 | | 6.3 |
| Kerikeri, NZ | Feb. 14, 2017 | Samson | 7.0 | | 24.9 |
| Ruakura, NZ | Feb. 20, 2017 | Samson | 8.1 | | 23.2 |
| Ballarat, AU | Feb. 23, 2017 | Samson | 4.7 | | 8.8 |
| Ruakura, NZ | Mar. 21, 2017 | Samson | 4.4 | | 10.9 |
| Ballarat, AU | Mar. 27, 2017 | Samson | 9.3 | | 17.1 |
| Kerikeri, NZ | Mar. 27, 2017 | Samson | 3.3 | | 27.3 |
| Lincoln, NZ | Feb. 7, 2018 | Samson | 16.2 | | 20.4 |
| Lincoln, NZ | Mar. 26, 2018 | Samson | 27.4 | | 36.0 |
| Lincoln, NZ | Jan. 28, 2019 | Samson | 13.8 | | 21.8 |
| Ruakura, NZ | Feb. 4, 2019 | Platform | 12.0 | 12.0 | 34.1 |
| Ruakura, NZ | Feb. 4, 2019 | Samson | 16.1 | 10.6 | 27.1 |
| Lincoln, NZ | Feb. 7, 2019 | Asset | 6.9 | 5.8 | 12.6 |
| Ruakura, NZ | Feb. 7, 2019 | Asset | 5.1 | 2.7 | 13.4 |
| Lincoln, NZ | Feb. 7, 2019 | Platform | 32.9 | 29.7 | 40.8 |
| Lincoln, NZ | Feb. 7, 2019 | Samson | 23.3 | 26.9 | 41.6 |
| Lincoln, NZ | Feb. 25, 2019 | Samson | 33.1 | | 41.7 |
| Lincoln, NZ | Mar. 18, 2019 | Samson | 41.0 | | 45.7 |
| Lincoln, NZ | Feb. 3, 2020 | Asset | 24.4 | 30.6 | 35.0 |
| Lincoln, NZ | Feb. 3, 2020 | Platform | 30.9 | 41.2 | 42.7 |
| Lincoln, NZ | Feb. 3, 2020 | Samson | 25.5 | 34.4 | 38.7 |
| Lincoln, NZ | Feb. 25, 2020 | Asset | 26.1 | 47.2 | 45.6 |
| Lincoln, NZ | Feb. 25, 2020 | Platform | 35.7 | 42.4 | 47.5 |
| Lincoln, NZ | Feb. 25, 2020 | Samson | 26.6 | 38.9 | 34.3 |
| Lincoln, NZ | Mar. 4, 2020 | Asset | 24.8 | 45.5 | 56.5 |
| Lincoln, NZ | Mar. 4, 2020 | Platform | 47.6 | 44.8 | 50.1 |
| Lincoln, NZ | Mar. 4, 2020 | Samson | 26.0 | 32.4 | 31.7 |

TABLE 6
Epoxyjanthitrem I production during the months of
January to March for a range of cultivar-endophyte combinations
at sites in New Zealand (NZ) and Australia (AU).

| | | | Epoxyjanthitrem I (ppm) | | |
|---|---|---|---|---|---|
| Sampling Site | Sampling Date | Cultivar | AR128 | AR166 | AR37 |
| Lincoln, NZ | Mar. 1, 2016 | Samson | 1.5 | | 4.4 |
| Kerikeri, NZ | Feb. 14, 2017 | Samson | 1.8 | | 7.8 |
| Ruakura, NZ | Feb. 20, 2017 | Samson | 2.5 | | 9.0 |
| Ballarat, AU | Feb. 23, 2017 | Samson | 1.5 | | 3.2 |
| Ruakura, NZ | Mar. 21, 2017 | Samson | 1.3 | | 4.0 |
| Ballarat, AU | Mar. 27, 2017 | Samson | 2.7 | | 6.2 |
| Kerikeri, NZ | Mar. 27, 2017 | Samson | 0.8 | | 8.6 |
| Lincoln, NZ | Feb. 7, 2018 | Samson | 4.6 | | 7.8 |
| Lincoln, NZ | Mar. 26, 2018 | Samson | 6.2 | | 9.0 |
| Lincoln, NZ | Jan. 28, 2019 | Samson | 3.9 | | 8.0 |
| Ruakura, NZ | Feb. 4, 2019 | Platform | 3.1 | 3.1 | 12.4 |
| Ruakura, NZ | Feb. 4, 2019 | Samson | 4.1 | 2.7 | 9.4 |
| Lincoln, NZ | Feb. 7, 2019 | Asset | 1.6 | 1.4 | 3.8 |
| Ruakura, NZ | Feb. 7, 2019 | Asset | 1.2 | 0.7 | 4.2 |
| Lincoln, NZ | Feb. 7, 2019 | Platform | 9.3 | 8.8 | 14.8 |
| Lincoln, NZ | Feb. 7, 2019 | Samson | 6.5 | 7.7 | 14.8 |
| Lincoln, NZ | Feb. 25, 2019 | Samson | 9.5 | | 14.8 |
| Lincoln, NZ | Mar. 18, 2019 | Samson | 10.0 | | 13.6 |
| Lincoln, NZ | Feb. 3, 2020 | Asset | 6.8 | 9.5 | 12.0 |
| Lincoln, NZ | Feb. 3, 2020 | Platform | 9.4 | 12.8 | 15.5 |
| Lincoln, NZ | Feb. 3, 2020 | Samson | 7.8 | 10.2 | 14.1 |
| Lincoln, NZ | Feb. 25, 2020 | Asset | 6.3 | 13.6 | 14.0 |
| Lincoln, NZ | Feb. 25, 2020 | Platform | 9.8 | 12.7 | 16.1 |
| Lincoln, NZ | Feb. 25, 2020 | Samson | 7.4 | 11.5 | 11.7 |
| Lincoln, NZ | Mar. 4, 2020 | Asset | 5.9 | 12.8 | 17.0 |
| Lincoln, NZ | Mar. 4, 2020 | Platform | 12.9 | 13.3 | 17.1 |
| Lincoln, NZ | Mar. 4, 2020 | Samson | 6.9 | 9.3 | 10.8 |

### Example 8

### Animal Safety Grazing Evaluation

### Background

An animal safety grazing evaluation trial of the epoxyjanthitrem-producing endophytes-AR128 and AR166—was undertaken in 2019/20 at AgResearch Lincoln. This trial included: AR37, AR128 and AR166 in association with each of cultivars, Samson diploid perennial ryegrass (Lolium perenne), Platform long-rotation diploid hybrid ryegrass (L. boucheanum), and Asset diploid Italian ryegrass (L. multiflorum). The positive (toxic) and negative controls were Samson infected with wild-type and AR1 endophytes, respectively (summarised in Table).

TABLE 7
Species of ryegrass cultivars used.

| Cultivar | Species | | Level of |
|---|---|---|---|
| name | common name | Species name | ploidy |
| Samson ^{A} | Perennial ryegrass | Lolium perenne | Diploid |
| Platform | Hybrid ryegrass | Lolium | Diploid |
| | | boucheanum B | |
| Asset | Italian ryegrass | Lolium multiflorum | Diploid |

^{A} Full cultivar name is ‘Grasslands Samson’.
^{B} synonymous with Lolium × hybridum

### Methods

The methodology was based in part on protocols previously used by Fletcher et al. (2017). These protocols have been developed and modified over many years in the evaluation of novel grass-endophyte associations for animal safety (Thom et al., 2012).

The 11 treatments were sown in three replicated pasture plots of 0.175 ha each. Drilling of pastures occurred over a 10-day period from 23 Sep. 2019. Pastures established well and were managed with applications of broadleaf herbicide, nitrogen fertiliser and irrigation during the spring and summer, in addition to light mechanical toppings. This ensured pure ryegrass pastures with standardised herbage mass were available prior to the commencement of the grazing trial in late summer. Ryegrass tillers (50 per plot) were collected mid-January 2020 for immuno-blotting to confirm the rates of endophyte infection (Simpson et al., 2012). Infection rates were very good, with all plots being in the 90-100% range. This confirmed all treatments exceeded the minimum rate of endophyte infection required for a valid test.

Prior to beginning any field work, Animal Ethics approval was sought and granted to allow the use of animals in this research (Invermay AE Committee, application #14822).

Weaned Romney ewe lambs (3-4 months old) were shorn, drenched to control internal parasites and dipped to prevent flystrike (myiasis) prior to being made available for the trial. Lambs were weighed to identify an even group of sheep, from which mobs of ten lambs with similar mean weights were then allocated to each plot. Mean lamb live weight was 27.0-28.3 kg at the beginning of the trial.

Plots were stocked with lambs on 5 Feb. 2020 (day 0) for replicate 1 and 6 Feb. 2020 (day 1) for replicates 2 and 3. The earliest symptoms of ryegrass staggers were observed in lambs on the wild-type treatment on day 5, so ryegrass staggers scoring began with replicate one on day 6, with replicates 2 and 3 being scored on each successive day (hence forth collectively referred to as day 7). Ryegrass staggers scoring involved running individual mobs of lambs 400 m along a laneway (Keogh 1973). Lambs showing a marked lack of coordination such that they could not complete the run were scored a 4. The remaining lambs were penned to assess the tremors associated with ryegrass staggers from scores 0 (no tremors) to 3 (marked tremors and some lack of coordination). Lambs that scored a 4 were removed from the trial for ethical reasons and replaced with spare lambs to maintain grazing pressure. For the purpose of comparing treatments, lambs removed due to severe ryegrass staggers (score=4) maintained their staggers score in the dataset for the remainder of the trial. Ryegrass staggers scoring then continued at 2-3 times each week. In total, lambs that remained for the full 40 days of the trial were scored 13 times for ryegrass staggers.

Ryegrass herbage samples cut at ground level were taken from 10 locations in each plot 2 days before plots were stocked (day-2), on day 20 and on day 28. Samples were frozen overnight and sent to the AgResearch Palmerston North for freeze drying, preparation and alkaloid analysis. Herbage was analysed for peramine, ergovaline, lolitrem B and epoxyjanthitrems, by minor modifications of established methods (Fletcher et al., 2017).

### Results

### Ryegrass Staggers

Ryegrass staggers developed rapidly, with the symptoms first identified on the Samson wild-type (toxic) control on day 5 (FIG. 4). By day 26, all lambs on wild-type plots had been removed due to suffering severe ryegrass staggers. This treatment easily met the minimum of >2.3 mean staggers score that is required for a trial to be valid.

Samson ryegrass (FIG. 4). Within the Samson treatments, notably in the first 15 days, the wild-type toxic control showed the greatest ryegrass staggers and by day 26 all lambs exhibited severe symptoms. Lambs grazing AR128 showed significantly lower stagger scores than those grazing wild-type or AR37.

Platform ryegrass (FIG. 5). As staggers increased, lambs grazing both AR128 and AR166 showed lower stagger scores than AR37 in the first 15 days of trial. Lambs grazing on AR128 maintained significantly lower stagger scores throughout the trial.

Asset ryegrass (FIG. 6). Lambs grazing on either AR128 or AR166 tended to show lower stagger scores compared to AR37 as the trial progressed.

Combined cultivars (FIG. 7). In a combined analysis across cultivars, the ryegrass staggers score of lambs grazing each of the AR128, AR166 and AR37 endophytes was calculated from measurements up until day 28 while all treatments were still being grazed. Throughout the course of the trial, ryegrass stagger scores in lambs grazing AR128 and AR166 infected cultivars trended lower than stagger scores in AR37, particularly so for AR128.

In summary, we have shown two alternative epoxyjanthitrem producing endophytes that provide better animal health results. Both AR128 and AR166 showed an overall trend of lower stagger scores than AR37, this is significantly noticeable in AR128.

### Endophyte Alkaloid Analysis

Herbage samples were collected in three harvests. Herbage samples collected at the beginning of the trial were analysed for ergovaline, lolitrem B, peramine and epoxyjanthitrems for all treatments. This confirmed there was no endophyte contamination of treatments or controls, and peramine concentrations of the Samson AR1 and wild-type controls were within expected values (mean of ~33 ppm). The second and third harvest samples were analysed for the alkaloids that related to the specific endophytes. For the wild-type control averaged over all harvests, ergovaline concentration was 1.0 ppm and lolitrem B concentration was 3.0 ppm-again within expected values for this type of trial, and at concentrations that would be toxic to animals.

AR128, AR166 and AR37 samples were analysed for epoxyjanthitrems from the three harvests (Table and Table).

Over the three cultivars and three harvest dates, mean total epoxyjanthitrem concentrations ranged from approximately 32 to 43 ppm (Table), while the mean epoxyjanthitrem I concentrations ranged from approximately 10 to 13 ppm (Table). On average, AR128 had lower concentrations of total epoxyjanthitrems and epoxyjanthitrem I than AR37 by 30% and 43%, respectively. On average, AR166 had lower concentrations of total epoxyjanthitrems and epoxyjanthitrem I than AR37 by 6% and 18%, respectively.

TABLE 8
Mean total epoxyjanthitrem concentration (ppm) and relative concentration to AR37 of
herbage samples taken during the grazing period. Means within columns followed by
different letters are significantly different (P ≤ 0.05).

| | Cultivar Samson | | | Cultivar Platform | | | Cultivar Asset | | | Mean of all cultivars | | |
|---|---|---|---|---|---|---|---|---|---|---|---|---|
| | | | Relative | | | Relative | | | Relative | | | Relative |
| | | | to AR37 | | | to AR37 | | | to AR37 | | | to AR37 |
| Endophyte | ppm | | (100%) | ppm | | (100%) | ppm | | (100%) | ppm | | (100%) |
| AR128 | 26.1 | b | 75% | 38.1 | b | 81% | 25.1 | b | 55% | 29.8 | b | 70% |
| AR166 | 35.3 | a | 101% | 42.8 | ab | 92% | 41.1 | a | 90% | 39.7 | a | 94% |
| AR37 | 34.9 | a | 100% | 46.8 | a | 100% | 45.7 | a | 100% | 42.5 | a | 100% |
| Mean | 32.1 | | | 42.6 | | | 37.3 | | | 37.3 | | |

TABLE 9
Mean epoxyjanthitrem I concentration (ppm) for ryegrass herbage samples taken during the grazing
period. Means within columns followed by different letters are significantly different (P ≤ 0.05).

| | Cultivar Samson | | | Cultivar Platform | | | Cultivar Asset | | | Mean of all cultivars | | |
|---|---|---|---|---|---|---|---|---|---|---|---|---|
| | | | Relative | | | Relative | | | Relative | | | Relative |
| | | | to AR37 | | | to AR37 | | | to AR37 | | | to AR37 |
| Endophyte | ppm | | (100%) | ppm | | (100%) | ppm | | (100%) | ppm | | (100%) |
| AR128 | 7.4 | b | 61% | 10.7 | c | 66% | 6.3 | c | 44% | 8.1 | c | 57% |
| AR166 | 10.3 | a | 84% | 12.9 | b | 80% | 12.0 | b | 83% | 11.7 | b | 82% |
| AR37 | 12.2 | a | 100% | 16.2 | a | 100% | 14.4 | a | 100% | 14.3 | a | 100% |
| Mean | 10.0 | | | 13.3 | | | 10.9 | | | 11.4 | | |

### Discussion

Within just a week of stocking plots, lambs grazing the Samson wild-type control exhibited symptoms of ryegrass staggers indicating that environmental conditions were highly conducive for the production of toxic levels of endophyte alkaloids along with greater staggers than what is usually experienced at this trial site using this methodology. The staggers in the well-characterised AR37 was also high, and similar to what has been reported in some years relative to staggers for wild-type (Fletcher and Sutherland 2009). The high level of staggers in wild-type, combined with high rates of tiller infection in all treatments and separation between controls (wild-type and AR1), confirmed that this grazing trial was a valid test for animal safety.

On average, ryegrass infected with AR128 and AR166 had lower concentrations of total epoxyjanthitrems and epoxyjanthitrem I than AR37, in line with what has also been found at other trial sites (see Example 7). As total epoxyjanthitrems, and in particular epoxyjanthitrem I, are known to cause staggers (Finch et al., 2020), these lower concentrations corresponded with the overall lower ryegrass staggers in lambs grazing ryegrass infected with AR128 and AR166, compared with AR37. Ryegrasses infected with AR128 or AR166 therefore present the opportunity to reduce ryegrass staggers compared to the equivalent ryegrasses infected with AR37.

### Example 9

### Effect of AR128 and AR166 Endophytes on Insect Pests

The AR128 Epichloë endophyte described herein entered the insect testing phase in 2015-16. The AR166 Epichloë endophyte entered insect testing in 2016-17. Here we report the efficacy of these endophytes against major insect pests.

We tested these different endophytes against Argentine stem weevil (Listronotus bonariensis), black beetle (Heteronychus arator), porina (Wiseana cervinata or W. copularis) and root aphid (Aploneura lentisci). Argentine stem weevil, black beetle adults and root aphid were each tested in replicated pot trials. Porina was tested in a replicated trial using freeze-dried ryegrass in an agar-based diet that provided epoxyjanthitrems at concentrations that commonly occur in planta. As control treatments, endophyte-free (Nil) and AR37-infected ryegrass plants were used as the basis for comparison. Other endophytes were also included in some trials. Because AR166 entered the testing pipeline one year later than AR128, some trials did not compare all epoxyjanthitrem-producing endophytes at once, but the two controls (endophyte-free (Nil) and AR37) were always included in all trials. All trials used perennial ryegrass cultivar Samson (Lolium perenne) as the test host grass, with Epichloë infection rates of 100% in endophyte-infected treatments and 0% for Nil plants.

For both Argentine stem weevil and black beetle trials, insects were caged onto ryegrass plants for up to 2 weeks. Adult weevil feeding and egg laying (oviposition) was recorded, and the cage covers were removed. Larval damage to ryegrass tillers (‘branches’ of ryegrass plant) was determined at approximately 3 weeks, with extent of damage scored as: 1=‘minor damage’ to the outside of the tiller; 2=‘moderate damage’, where the larva had penetrated and partially mined the tiller; and 3=‘severe damage’, where the larva had extensively mined the tiller and/or the meristem (tiller growing point) had been destroyed. Black beetle adult feeding was scored between 1-2 weeks after caging, on the same scale as for the Argentine stem weevil but was based on the damage to the very base of tillers.

Root aphid populations were determined by counting the number of colonies on the outer surface of root mass of the soil/sand mix that was used as a potting medium. Colonies may comprise 1 or several aphids that have surrounded themselves in white flocculent wax.

Porina diet consumption and weight gain were measured in a plant assay in which larvae of known weight were fed an agar-based diet in which ground freeze-dried ryegrass had been incorporated. The ryegrass was harvested from potted plants in June 2017 and was comprised of pseudostems and leaf blades cut at a height of between 2 and 6 cm. Diets were prepared weekly, and then changed at 3 days with new diet supplied after a further 4 days. The trial continued for 3 weeks. Diets were weighed at the beginning and end of each feeding period to determine diet consumption, while weight of larvae was determined at the start and end of the trial. The dry weight epoxyjanthitrem concentrations in the herbage used in diets were measured.

### Argentine Stem Weevil

Trial 1: Endophyte treatments were AR128, AR37 and Nil. Adult feeding, the number of eggs laid (oviposition), and larval damage were measured (Error! Reference source not found.).

Adult feeding did not differ significantly between the two epoxyjanthitrem-producing endophytes. The number of eggs laid per plant followed a similar pattern to adult feeding with no difference between the two epoxyjanthitrem-producing endophytes.

TABLE 10
Argentine stem weevil adult feeding and oviposition
(egg laying), and larval damage, on perennial ryegrass
infected with epoxyjanthitrem-producing endophytes (AR37,
AR128), and an endophyte-free (Nil)
control in Trial 1. Within columns, means
followed by the same letter are not significantly different (P ≤ 0.05).

| | Adult feeding & oviposition | | Larval damage (% of tillers) | |
|---|---|---|---|---|
| Endophyte | No. | No. eggs/ | All levels | Moderate & |
| treatment | scars/tiller | plant | of damage | severe damage |
| AR128 | 22.3 a | 22.8 a | 23.5 b | 13.5 b |
| AR37 | 23.4 a | 25.5 a | 31.0 b | 15.8 b |
| Nil | 24.4 a | 28.9 a | 65.2 a | 60.4 a |
| LSD 5% | 5.57 | 14.51 | 13.15 | 12.79 |

The percentage of tillers with all levels of larval damage, and most importantly those with moderate and severe damage, was similar across all endophyte-infected treatments and all had significantly less damage than Nil (P<0.001). The table demonstrates that the mode of action for the epoxyjanthitrem-producing endophytes is suppression of larval damage.

Trial 2: AR166 was included in a trial testing other endophytes, along with AR37 and a Nil control (Error! Reference source not found.). AR166 and AR37 had reduced percentage of t illers with all levels of Argentine stem weevil larval damage (P=0.002), and most importantly the moderate and severe damage (P<0.001) to very low levels, compared with Nil.

TABLE 11
Percentage of tillers with Argentine stem weevil larval damage
on perennial ryegrass infected with two epoxyjanthitrem-
producing endophytes (AR166, AR37) compared with
an endophyte-free (Nil) control in Trial 2. Within columns, means
followed by the same letter are not significantly different (P ≤ 0.05).

| | Larval damage (% of tillers) | |
|---|---|---|
| Endophyte | | Moderate & severe |
| treatment | All levels of damage | damage |
| AR166 | 3.6 b | 1.6 b |
| AR37 | 8.5 b | 3.5 b |
| Nil | 27.7 a | 25.7 a |
| LSD 5% | 12.3 | 9.79 |

In summary, the data from these two trials provide robust evidence that epoxyjanthitrem-producing endophytes (AR128, AR166, AR37) provide strong protection from the most damaging life stage (i.e. larvae) of the Argentine stem weevil. Also this protection is carried through to the reduction in moderate to severe tiller damage, these types of damages are significant as they impact the survival and growth of the plant.

### Black Beetle

Trial 1: This trial compared two epoxyjanthitrem-producing endophytes (AR37, AR128) with an endophyte-free (Nil) control. Both endophytes significantly reduced the percentage of tillers with severe damage compared with endophyte-free (P=0.001) (Error! Reference source not found.).

TABLE 12
Percentage of ryegrass tillers with severe damage by adult black
beetle, comparing two epoxyjanthitrem-producing endophytes
(AR37, AR128) with an endophyte-free (Nil) control in
Trial 1. Means followed by the same letter
are not significantly different (P<0.05).

| | Adult black beetle |
|---|---|
| Endophyte | damage (% of tillers) |
| treatment | Severe damage |
| AR128 | 21.7 b |
| AR37 | 11.2 b |
| Nil | 42.8 a |
| LSD 5% | 15.35 |

Trial 2: This trial compared a peramine-producing endophyte (AR1) and three epoxyjanthitrem-producing endophytes (AR37, AR128, AR166), with an endophyte-free (Nil) control. All endophytes significantly reduced the percentage of tillers with all levels of damage, and most importantly moderate and severe damage, compared with Nil (P<0.001) (Error! Reference source not found.).

Only AR166 had significantly less damage than AR1, but damage to plants with this endophyte was not significantly different from plants infected with AR37 and AR128 in this trial.

TABLE 13
Percentage of ryegrass tillers with all levels of
damage by adult black beetle and the percentage of tillers
with moderate and severe damage by adult
black beetle, comparing a peramine-producing endophyte
(AR1) and three epoxyjanthitrem-producing endophytes (AR37,
AR128, AR166), with an endophyte-free (Nil)
control in Trial 2. Within columns, means followed
by the same letter are not significantly different (P ≤ 0.05).

| | Adult black beetle damage | |
|---|---|---|
| | (% of tillers) | |
| Endophyte | | Moderate & severe |
| treatment | All levels of damage | damage |
| AR1 | 32.4 b | 31.2 b |
| AR128 | 25.4 bc | 22.7 bc |
| AR166 | 14.9 c | 12.7 c |
| AR37 | 23.5 bc | 21.4 bc |
| Nil | 53.1 a | 52.3 a |
| LSD 5% | 14.92 | 14.53 |

In summary, the data from these two trials provide robust evidence that epoxyjanthitrem-producing endophytes (AR128, AR166, AR37) provide good protection from adult black beetle.

### Root Aphid

Trial 1: In this trial, aphid colonies on the roots growing on the outer surface of the root mass were counted. There were significant (P<0.001) differences between the endophyte treatments in this trial (Table). The two epoxyjanthitrem-producing endophytes (AR128, AR37) significantly reduced aphid infestations compared with endophyte-free (Nil).

TABLE 14
Number of root aphid colonies (and log transformed
data) on the outer roots of potted ryegrass
plants infected with epoxyjanthitrem-producing
endophytes (AR128, AR37) and an endophyte-free
control (Nil), in Trial 1. Within columns, means followed by
the same letter are not significantly different (P≤0.05).

| Endophyte | | |
|---|---|---|
| treatment | No. colonies | Log no. colonies |
| AR128 | 1.6 b | 0.174 b |
| AR37 | −0.2 b | 0.069 b |
| Nil | 26.6 a | 1.136 a |
| LSD 5% | 12.87 | 0.2857 |

Trial 2: As for Trial 1, colony counts were done on the outer roots of each plant in a trial that included AR166. There were significant (P<0.001) differences between treatments (Table). Both epoxyjanthitrem-producing endophytes (AR166 and AR37) significantly reduced aphid infestations compared with endophyte-free (Nil).

TABLE 15
Number of root aphid colonies (log transformed data)
found on the outer roots of potted ryegrass plants infected
with epoxyjanthitrem-producing endophytes
(AR166, AR37), compared with a peramine-producing endophyte
(AR1) and an endophyte-free control (Nil), in Trial 2. Means
followed by the same letter are not significantly different (P ≤ 0.05).

| Endophyte | Log No. |
|---|---|
| treatment | Colonies |
| AR1 | 0.414 b |
| AR166 | 0.081 bc |
| AR37 | 0.025 c |
| Nil | 0.864 a |
| LSD 5% | 0.3533 |

In summary, the data from these two trials provide robust evidence that epoxyjanthitrem-producing endophytes (AR128, AR166, AR37) provide good protection from root aphid.

### Porina

In this assay, freeze-dried plant material was incorporated into an agar-based diet and fed to porina larvae. AR128 and AR166 endophytes were compared with AR37 and endophyte-free (Nil) for their effects on consumption and growth of porina larvae over 2 weeks (Table). Compared with Nil, endophytes AR128, AR166 and AR37 significantly reduced feeding and weight gain, with the effects of AR166 and AR37 being significantly greater than those of AR128.

TABLE 16
Comparison of total amount of porina larvae diet consumed for diets containing freeze-
dried ryegrass infected with AR37, AR128 and AR166 with an endophyte-free (Nil) control, the
associated weight change of larvae, and epoxyjanthitrem concentrations in the ryegrass herbage
incorporated into the diets (ppm dry weight). Within rows, means followed by the same letter are
not significantly different (P ≤ 0.05).

| | Endophyte Treatment | | | | |
|---|---|---|---|---|---|
| | AR128 | AR166 | AR37 | Nil | LSD 5% |
| Mean consumption (mg) | 361 b | 97 c | 70 c | 1254 a | 145.6 |
| Mean weight change | 18.5 b | −21.9 c | −12.6 c | 75.3 a | 14.6 |
| (mg) | | | | | |
| Epoxyjanthitrem | 14.01 | 68.79 | 52.33 | | |
| concentration in herbage | | | | | |
| (ppm) | | | | | |

In summary, the data from this trial provides good evidence that epoxyjanthitrem-producing endophytes (AR128, AR166, AR37) provide good protection from porina larvae.

Overall, our example shows that across the range of insects tested, AR128 and AR166 had a significant impact on insect damage and insect diet consumption compared to Nil endophyte.

Disclosed herein is the inventors surprising determination that two Epichloë endophyte strains produce, in planta, lower total epoxyjanthitrem levels, and epoxyjanthitrem I levels as compared to the known Epichloë endophyte AR37. AR37 is known to impart good insect resistance to an infected host plant. However, livestock animals grazing on host plant/AR37 combinations can experience animal toxicosis, including ryegrass staggers. The work detailed herein provides two endophyte strains, which, as shown in our examples, have distinct and unexpected advantages over known Epichloë strains, particularly in showing a marked reduction in Epichloë toxicosis as measured by ryegrass stagger scores. This result is achieved without compromising insect resistance. Without wishing to be bound by theory, the inventors believe that the basis of their unexpected achievement is found in the reduced levels of total epoxyjanthitrems and epoxyjanthitrem I produced by the Epichloë endophytes of the invention.

Although the invention has been described by way of example and with reference to particular embodiments, it is to be understood that modifications and/or improvements may be made without departing from the scope of the invention.

In addition, where features or aspects of the invention are described in terms of Markush groups, those skilled in the art will recognise that the invention is also thereby described in terms of any individual member or subgroup of members of the Markush group.

### INDUSTRIAL APPLICATION

The isolated Epichloë endophyte strains, plant/Epichloë endophyte combinations, seeds infected with Epichloë endophytes, and methods of making and using such combinations according to the invention as disclosed herein all have industrial application for the production of plants that are used for human or animal consumption.

### REFERENCES

Andrews S. 2010. FastQC: a quality control tool for high throughput sequence data. https://www.bioinformatics.babraham.ac.uk/projects/fastqo/Ball
O-J P, Miles C O, Prestidge R A. 1997a. Ergopeptine alkaloids and Neotyphodium lolii-mediated resistance in perennial ryegrass against adult Heteronychus arator (Coleoptera: Scarabaeidae). Journal of Economic Entomology 90:1382-1391.
Bolger A M, Lohse M, Usadel B. 2014. Trimmomatic: a flexible trimmer for Illumina sequence data. Bioinformatics 30 (15): 2114-2120.
Brownstein M J, Carpten J D, Smith J R (1996) Modulation of non-templated nucleotide addition by Taq DNA polymerase: Primer modifications that facilitate genotyping. BioTechniques 20:1004-1010.
Card S D, Faville M J, Simpson W R, Johnson R D, Voisey C R, de Bonth ACM, Hume D E. 2014. Mutualistic fungal endophytes in the Triticeae-survey and description. FEMS Microbiol Ecology 88:94-106.
Christensen M J, Leuchtmann A, Rowan D D, Tapper B A. 1993. Taxonomy of Acremonium endophytes of tall fescue (Festuca arundinacea), meadow fescue (F. pratensis) and perennial ryegrass (Lolium perenne). Mycological Research 97:1083-92.
Christensen M J, Bennett R J, Schmid J. 2002. Growth of Epichloë/Neotyphodium and p-endophytes in leaves of Lolium and Festuca grasses. Mycological Research 106:93-106.
Danecek P, Auton A, Abecasis G, Albers C A, Banks E, DePristo M A, Handsaker R E, Lunter G, Marth G T, Sherry S T, McVean G, Durbin R, Genomes Project Analysis Group. 2011. The variant call format and VCFtools. Bioinformatics 27 (15): 2156-2158.
Dymock J J, Prestidge R A, Rowan D D. 1989. The effects of lolitrem B on Argentine stem weevil larvae. Proceedings of the New Zealand Plant Protection Conference 42:73-75.
Finch S C, Prinsep M R, Popay A J, Wilkins A L, Webb N G, Bhattarai S, Jensen J G, Hawkes A D, Babu J V, Tapper B A, Lane G A. 2020. Identification and structure elucidation of epoxyjanthitrems from Lolium perenne infected with the endophytic fungus Epichloë festucae var. lolii and determination of the tremorgenic and anti-insect activity of epoxyjanthitrem I. Toxins 12.
Fletcher L R, Sutherland B L. 2009. Sheep responses to grazing ryegrass with AR37. Proceedings of the New Zealand Grassland Association 71:127-132.
Fletcher L R, Finch S C, Sutherland B L, deNicolo G, Mace W J, Van Koten C, Hume D E. 2017. The occurrence of ryegrass staggers and heat stress in sheep grazing ryegrass-endophyte associations with diverse alkaloid profiles. New Zealand Veterinary Journal 65:232-41.
Gallagher R T, White E P, Mortimer P H. 1981. Ryegrass staggers: isolation of potent neurotoxins lolitrem A and lolitrem B from staggers-producing pastures. New Zealand Veterinary Journal 29:189-190.
Gallagher R T, Smith G S, di Menna M E, Young P W. 1982. Some observations on neurotoxin production in perennial ryegrass. New Zealand Veterinary Journal 30:203-204.
Glenn A E, Bacon C W, Price R, Hanlin R T. 1996. Molecular phylogeny of Acremonium and its taxonomic implications. Mycologia 88:369-83.
Hettiarachchige I K, Ekanayake P N, Mann R C, Guthridge K M, Sawbridge T I, Spangenberg G C, Forster J W. 2015. Phylogenomics of asexual Epichloë fungal endophytes forming associations with perennial ryegrass. BMC Evolutionary Biology 15:72.
Hume D E, Ryan D L, Cooper B M, Popay A J. 2007. Agronomic performance of AR37-infected ryegrass in northern New Zealand. Proceedings of the New Zealand Grassland Association 69:201-5.
Keogh R G. 1973. Induction and prevention of ryegrass staggers in grazing sheep. New Zealand Journal of Experimental Agriculture 1:55-57.
Kirkby K A, Pratley J E, Hume D E, Faville M J, An M, Wu H. 2011. Incidence of endophyte Neotyphodium occultans in Lolium rigidum from Australia. Weed Research 51:261-272.
Koren S, Walenz B P, Berlin K, Miller J R, Bergman N H, Phillippy A M. 2017. Canu: scalable and accurate long-read assembly via adaptive k-mer weighting and repeat separation. Genome Research, gr-215087.
Koulman A, Lane G A, Christensen M J, Fraser K, Tapper B A. 2007. Peramine and other fungal alkaloids are exuded in the guttation fluid of endophyte-infected grasses. Phytochemistry 68:355-360.
Latch G C M, Christensen M J. 1985. Artificial infection of grasses with endophytes. Annals of Applied Biology 107:17-24.
Leuchtmann A, Bacon C W, Schardl C L, White Jr J F, Tadych M. 2014. Nomenclatural realignment of Neotyphodium species with genus Epichloë. Mycologia 106:202-215.
Li H, Durbin R. 2009. Fast and accurate short read alignment with Burrows-Wheeler transform. Bioinformatics 25:1754-1760.
Li H, Handsaker B, Wysoker A, Fennell T, Ruan J, Homer N, Marth G, Abecasis G, Durbin R, Subgroup GPDP. 2009. The sequence alignment/map format and SAMtools. Bioinformatics 25:2078-2079.
Li H. 2011. A statistical framework for SNP calling, mutation discovery, association mapping and population genetical parameter estimation from sequencing data. Bioinformatics 27:2987-2993.
Moon C D, Tapper B A, Scott B. 1999. Identification of Epichloë endophytes in planta by a microsatellite-based PCR fingerprinting assay with automated analysis. Applied and Environmental Microbiology 65:1268-1279.
Mortimer P H, Fletcher L R, Menna MEd, Harvey I C, Smith G S, Barker G M, Gallagher R T, White E P. 1982. Recent advances in ryegrass staggers. Proceedings of the Ruakura Farmers' Conference 34:71-74.
Oliveros J C. 2007. Venny. https://bioinfogp.cnb.csic.es/tools/venny/.
Popay A J, Hume D E. 2011. Endophytes improve ryegrass persistence by controlling insects. In: Pasture Persistence Symposium. Grassland Research and Practice Series No. 15 (ed C. F. Mercer) (New Zealand Grassland Association, Dunedin) 149-156.
Prestidge R A, Pottinger R P, Barker G M. 1982. An association of Lolium endophyte with ryegrass resistance to Argentine stem weevil. Proceedings of the Thirty Fifth New Zealand Weed and Pest Control Conference 35:119-122.
Rowan D D, Dymock J J, Brimble M A. 1990. Effect of fungal metabolite peramine and analogs on feeding and development of Argentine stem weevil (Listronotus bonariensis). Journal of Chemical Ecology 16:1683-1695.
Schardl C L, Craven K D, Speakman S, Stromberg A, Lindstrom A, Yoshida R. 2008. A novel test for host-symbiont codivergence indicates ancient origin of fungal endophytes in grasses. Systematic Biology 57:483-498.
Schuelke M. 2000. An economic method for the fluorescent labelling of PCR fragments. Nature Biotechnology 18:233-234.
Simpson W R, Mace W J. 2012. Novel associations between Epichloë endophytes and grasses: Possibilities and outcomes. In ‘Epichloë, endophytes of cool season grasses: Implications, utilization and biology.’ (Eds C A Young, G E Aiken, R L McCulley, J R Strickland, C L Schardl.) pp. 35-39. (The Samuel Roberts Noble Foundation: Ardmore, Oklahoma).
Simpson W R, Schmid J, Singh J, Faville M J, Johnson R D. 2012. A morphological change in the fungal symbiont Neotyphodium lolii induces dwarfing in its host plant Lolium perenne. Fungal Biology 116:234-240.
Thom E R, Popay A J, Hume D E, Fletcher L R. 2012. Evaluating the performance of endophytes in farm systems to improve farmer outcomes-A review. Crop and Pasture Science 63:927-43.
Thorvaldsdóttir H, Robinson J T, Mesirov J P. 2013. Integrative Genomics Viewer (IGV): high-performance genomics data visualization and exploration. Briefings in Bioinformatics 14:178-192.
Zhang, D X, Nagabhyru P, Blankenship J D, Schardl C L. 2010. Are loline alkaloid levels regulated in grass endophytes by gene expression or substrate availability? Plant Signaling and Behavior 5:1419-22.

### Microorganism Deposits

### Description of the Microorganism Deposits Made Under the Budapest Treaty

The following biological deposits have been made under the terms of the Budapest Treaty on the International Recognition of the Deposit of Micro-organisms for the Purposes of Patent Procedure.

| Deposit Identification | International Depository | |
|---|---|---|
| Reference | Designation | Date of Deposit |
| AR 128 | NRRL 68010 | Dec. 23, 2020 |
| AR 166 | NRRL 68011 | Dec. 23, 2020 |

Certificates of Deposit and Statements of Viability for the above deposited micro-organisms are appended below.

1.-18. (canceled)

19. An artificial combination comprising a host plant that infected with an isolated strain of Epichloë endophyte wherein the endophyte comprises an ans017 allele size of 306±0.8 base pairs (bp), an ans030 allele size of 308±0.8 bp, an ans033 allele size of 171=0.8 bp, an ans054 allele size of 297±0.8 bp, an egs002 allele size of 297±0.8 bp and an egs027 allele size of 344±0.8 bp.

20. The combination of claim 19 wherein the Epichloë endophyte comprises at least one additional SSR allele selected from the group consisting of ans014, ans015, ans016, ans019, ans024, ans025, ans031, ans032, ans035, ans036, ans044, ans047, ans049, ans056, egs004, egs010 and egs027, wherein the at least one additional SSR allele has the number of base pairs (bp) as shown in Table 3, ±0.8 bp.

21. The combination of claim 19 wherein the Epichloë endophyte comprises at least two additional SSR alleles, preferably at least 3, at least 4, at least 5, at least 6, at least 7, at least 8, at least 9, at least 10, at least 11, at least 12, at least 13, at least 14, at least 15, at least 16, at least 17, at least 18 additional SSR alleles, preferably 19 additional SSR alleles, wherein each of the additional SSR alleles has the number of base pairs (bp) as shown in Table 3, ±0.8 bp.

22. The combination of claim 19 wherein the Epichloë endophyte comprises the following 22 SSR alleles: B10, B11, ans014, ans015, ans016, ans017, ans019, ans024, ans025, ans030, ans031, ans032, ans033, ans035, ans036, ans044, ans047, ans049, ans054, ans056, egs002, egs004, egs010 and egs027, wherein the 24 SSR alleles have the number of base pairs (bp) as shown in Table 3, ±0.8 bp.

23. The combination of claim 19 wherein the Epichloë endophyte is AR128 (NRRL 68010) or AR166 (NRRL 68011) or a combination thereof.

24. The combination of claim 19 wherein the Epichloë endophyte produces in planta about 2 ppm to about 60 ppm of epoxyjanthitrem compounds.

25. The combination of claim 24, wherein the about 2 ppm to about 60 ppm of epoxyjanthitrem compounds is produced in planta during austral summer.

26. The combination of claim 25 wherein the austral summer is January to March.

27. The combination of claim 19 wherein Epichloë endophyte does not produce, in planta, more than about 0.1 ppm ergovaline or more than about 0.1 ppm lolitrem B or both.

28. A host plant or part thereof that is artificially infected with the isolated strain of Epichloë endophyte claim 19.

29. The artificially infected host plant of claim 28 wherein the part thereof is a seed.

30. A method of conferring at least some level of pest protection on a host plant comprising artificially infecting the host plant with AR128 or AR166 to form a host plant/Epichloëendophyte combination.

31. A method of reducing the average level of Epichloë toxicosis experienced by an animal grazing on an Epichloë infected host plant comprising feeding the animal with a host plant that is artificially infected with AR128 or AR166, wherein the reduction in the average level of Epichloë toxicosis experienced by the animal is an average reduction relative to the average level of Epichloë toxicosis experienced by the same type of grazing animal feeding on a host plant that is infected with a selected or wild-type Epichloë endophyte.


SEQUENCE LISTING (50 sequences)

SEQ ID NO: 1 - DNA, 18 bp, Artificial Sequence
  Synthesized
tgtaaaacgacggccagt

SEQ ID NO: 2 - skipped

SEQ ID NO: 3 - DNA, 24 bp, Artificial Sequence
  Synthesized
cgctcagggctacatacaccatgg

SEQ ID NO: 4 - DNA, 23 bp, Artificial Sequence
  Synthesized
ctcatcgagtaacgcaggcgacg

SEQ ID NO: 5 - DNA, 24 bp, Artificial Sequence
  Synthesized
catggatggacaagagattgcacg

SEQ ID NO: 6 - DNA, 24 bp, Artificial Sequence
  Synthesized
ttcactgctacaattctgtccagc

SEQ ID NO: 7 - DNA, 22 bp, Artificial Sequence
  Synthesized
caatcattctgaaccaaaacca

SEQ ID NO: 8 - DNA, 22 bp, Artificial Sequence
  Synthesized
ccttatacgcaggaccaaagac

SEQ ID NO: 9 - DNA, 22 bp, Artificial Sequence
  Synthesized
gtctaaacagcacagagcgaga

SEQ ID NO: 10 - DNA, 22 bp, Artificial Sequence
  Synthesized
acccgagtcaagttgcattatt

SEQ ID NO: 11 - DNA, 21 bp, Artificial Sequence
  Synthesized
cacaaagacaaacgccaaaag

SEQ ID NO: 12 - DNA, 22 bp, Artificial Sequence
  Synthesized
gcaaagctcacagacaaaggtc

SEQ ID NO: 13 - DNA, 22 bp, Artificial Sequence
  Synthesized
gccatccgataacctgtctcta

SEQ ID NO: 14 - DNA, 22 bp, Artificial Sequence
  Synthesized
tcgtttcggttcgattaagagt

SEQ ID NO: 15 - DNA, 21 bp, Artificial Sequence
  Synthesized
tacctctgcacggtgtattcc

SEQ ID NO: 16 - DNA, 24 bp, Artificial Sequence
  Synthesized
tgcataacactcaccttatagtcg

SEQ ID NO: 17 - DNA, 22 bp, Artificial Sequence
  Synthesized
ctggactacctcctccacaatc

SEQ ID NO: 18 - DNA, 21 bp, Artificial Sequence
  Synthesized
gcccgcattaaagaaataagg

SEQ ID NO: 19 - DNA, 23 bp, Artificial Sequence
  Synthesized
cagccctattttatgtttgaagg

SEQ ID NO: 20 - DNA, 22 bp, Artificial Sequence
  Synthesized
gctcctgcttttattcctgcta

SEQ ID NO: 21 - DNA, 24 bp, Artificial Sequence
  Synthesized
gcataggaagtgctgttaatttga

SEQ ID NO: 22 - DNA, 22 bp, Artificial Sequence
  Synthesized
agagtagaacctgcttgcgtta

SEQ ID NO: 23 - DNA, 22 bp, Artificial Sequence
  Synthesized
actcggagacatgaaaaccatc

SEQ ID NO: 24 - DNA, 22 bp, Artificial Sequence
  Synthesized
gacgtgctctgtgatgttgaat

SEQ ID NO: 25 - DNA, 24 bp, Artificial Sequence
  Synthesized
gattcacctcatctaccatctacc

SEQ ID NO: 26 - DNA, 22 bp, Artificial Sequence
  Synthesized
ggtcgagtttatatctccgatg

SEQ ID NO: 27 - DNA, 22 bp, Artificial Sequence
  Synthesized
gcgttgaggaggctagatagaa

SEQ ID NO: 28 - DNA, 22 bp, Artificial Sequence
  Synthesized
ttccaagctgaacaaaagtcaa

SEQ ID NO: 29 - DNA, 22 bp, Artificial Sequence
  Synthesized
cggtgctctttaccttttgact

SEQ ID NO: 30 - DNA, 22 bp, Artificial Sequence
  Synthesized
catataagaggcccatctcacc

SEQ ID NO: 31 - DNA, 22 bp, Artificial Sequence
  Synthesized
atttgcagcagagatgatgtgt

SEQ ID NO: 32 - DNA, 22 bp, Artificial Sequence
  Synthesized
cctgcaccggactgttagtaat

SEQ ID NO: 33 - DNA, 22 bp, Artificial Sequence
  Synthesized
tccagaagtagtcgggatcagt

SEQ ID NO: 34 - DNA, 22 bp, Artificial Sequence
  Synthesized
atgagaggggaattgaagacaa

SEQ ID NO: 35 - DNA, 22 bp, Artificial Sequence
  Synthesized
tcacctctctcgacgttgtcta

SEQ ID NO: 36 - DNA, 22 bp, Artificial Sequence
  Synthesized
tctcgtaaaacccaattccatc

SEQ ID NO: 37 - DNA, 22 bp, Artificial Sequence
  Synthesized
atctcggctctaacaaagttcg

SEQ ID NO: 38 - DNA, 22 bp, Artificial Sequence
  Synthesized
gcatatacggacgtgctaccta

SEQ ID NO: 39 - DNA, 22 bp, Artificial Sequence
  Synthesized
cttgtgatgagaatcgtcaagc

SEQ ID NO: 40 - DNA, 22 bp, Artificial Sequence
  Synthesized
ccctaccgaaactacatcgttc

SEQ ID NO: 41 - DNA, 21 bp, Artificial Sequence
  Synthesized
agaccatcatgtgagcgactt

SEQ ID NO: 42 - DNA, 22 bp, Artificial Sequence
  Synthesized
gtgatgccctctatgtgtatgc

SEQ ID NO: 43 - DNA, 24 bp, Artificial Sequence
  Synthesized
taaggcgaggtcagttattagctt

SEQ ID NO: 44 - DNA, 24 bp, Artificial Sequence
  Synthesized
ctcttttgggacctacctagtcaa

SEQ ID NO: 45 - DNA, 24 bp, Artificial Sequence
  Synthesized
tatacagactacgaacaccctccc

SEQ ID NO: 46 - DNA, 24 bp, Artificial Sequence
  Synthesized
ggatgttactgaatgaaaactccc

SEQ ID NO: 47 - DNA, 24 bp, Artificial Sequence
  Synthesized
attccttgtcctgttgtctcaaac

SEQ ID NO: 48 - DNA, 24 bp, Artificial Sequence
  Synthesized
atggttgctcgaaaagacttactc

SEQ ID NO: 49 - DNA, 24 bp, Artificial Sequence
  Synthesized
gatgacgtatcttgatgctaccac

SEQ ID NO: 50 - DNA, 24 bp, Artificial Sequence
  Synthesized
cgtgtataaagttcgggatcctat